(19) United States
(12) Patent Application Publication
(10) Pub. No.: US 2004/0116866 A1
(43) Pub. Date: Jun. 17, 2004

(54) Skin attachment apparatus and method for patient infusion device

(76) Inventors: William Gorman, South Hamilton, MA (US); John T. Garibotto, Charlestown, MA (US); J. Christopher Flaherty, Topsfield, MA (US); Amy W. Cai, Cambridge, MA (US); Thomas J. Dragosits, North Andover, MA (US)

Correspondence Address: MCDERMOTT, WILL & EMERY, ATTN: INTELLECTUAL PROPERTY DEPT., 28 STATE STREET, BOSTON, MA, 02109, US

(21) Appl. No.: 10/321,268

(22) Filed: Dec. 17, 2002

(51) Int. Cl. A61M 5/32

(52) U.S. Cl. 604/174; 604/93.01

(57) ABSTRACT

An apparatus adapted to be disposed between a rigid or semi-rigid device and human skin for reliably attaching the device to the skin for an extended period of time. The apparatus includes a carrier having a skin-contacting surface, and an opposed device-contacting surface, and at least one skin adhesive layer secured to the skin-contacting surface of the carrier for securing the carrier to the skin. A surface area of the skin adhesive layer is less than an area of the skin-contacting surface of the carrier.

### CROSS-REFERENCE TO RELATED APPLICATIONS

The present application is related to co-pending U.S. patent application Ser. No. 09/943,992, filed on Aug. 31, 2001 (Atty. Docket No. INSL-110), and entitled DEVICES, SYSTEMS AND METHODS FOR PATIENT INFUSION, which is assigned to the assignee of the present application and incorporated herein by reference.

### FIELD OF THE INVENTION

The present invention relates generally to medical devices, systems and methods, and more particularly to small, low cost, portable infusion devices and methods that are useable to achieve precise, sophisticated, and programmable flow patterns for the delivery of therapeutic liquids such as insulin to a mammalian patient. Even more particularly, the present invention is directed to various new and improved skin attachment apparatuses and methods for an infusion device.

### BACKGROUND OF THE INVENTION

Today, there are numerous diseases and other physical ailments that are treated by various medicines including pharmaceuticals, nutritional formulas, biologically derived or active agents, hormonal and gene based material and other substances in both solid or liquid form. In the delivery of these medicines, it is often desirable to bypass the digestive system of a mammalian patient to avoid degradation of the active ingredients caused by the catalytic enzymes in the digestive tract and liver. Delivery of a medicine other than by way of the intestines is known as parenteral delivery. Parenteral delivery of various drugs in liquid form is often desired to enhance the effect of the substance being delivered, insuring that the unaltered medicine reaches its intended site at a significant concentration. Also, undesired side effects associated with other routes of delivery, such as systemic toxicity, can potentially be avoided.

Often, a medicine may only be available in a liquid form, or the liquid version may have desirable characteristics that cannot be achieved with solid or pill form. Delivery of liquid medicines may best be accomplished by infusing directly into the cardiovascular system via veins or arteries, into the subcutaneous tissue or directly into organs, tumors, cavities, bones or other site specific locations within the body.

Parenteral delivery of liquid medicines into the body is often accomplished by administering bolus injections using a needle and reservoir, or continuously by gravity driven dispensers or transdermal patch technologies. Bolus injections often imperfectly match the clinical needs of the patient, and usually require larger individual doses than are desired at the specific time they are given. Continuous delivery of medicine through gravity feed systems compromise the patient's mobility and lifestyle, and limit the therapy to simplistic flow rates and profiles. Transdermal patches have special requirements of the medicine being delivered, particularly as it relates to the molecular structure, and similar to gravity feed systems, the control of the drug administration is severely limited.

Ambulatory infusion pumps have been developed for delivering liquid medicaments to a patient. These infusion devices have the ability to offer sophisticated fluid delivery profiles accomplishing bolus requirements, continuous infusion and variable flow rate delivery. These infusion capabilities usually result in better efficacy of the drug and therapy and less toxicity to the patient's system. An example of a use of an ambulatory infusion pump is for the delivery of insulin for the treatment of diabetes mellitus. These pumps can deliver insulin on a continuous basal basis as well as a bolus basis as is disclosed in U.S. Pat. No. 4,498,843 to Schneider et al.

The ambulatory pumps often work with a reservoir to contain the liquid medicine, such as a cartridge, a syringe or an IV bag, and use electromechanical pumping or metering technology to deliver the medication to the patient via tubing from the infusion device to a needle that is inserted transcutaneously, or through the skin of the patient. The devices allow control and programming via electromechanical buttons or switches located on the housing of the device, and accessed by the patient or clinician. The devices include visual feedback via text or graphic screens, such as liquid crystal displays known as LCD's, and may include alert or warning lights and audio or vibration signals and alarms. The device can be worn in a harness or pocket or strapped to the body of the patient.

Currently available ambulatory infusion devices are expensive, difficult to program and prepare for infusion, and tend to be bulky, heavy and very fragile. Filling these devices can be difficult and require the patient to carry both the intended medication as well as filling accessories. The devices require specialized care, maintenance, and cleaning to assure proper functionality and safety for their intended long term use. Due to the high cost of existing devices, healthcare providers limit the patient populations approved to use the devices and therapies for which the devices can be used.

Clearly, therefore, there was a need for a programmable and adjustable infusion system that is precise and reliable and can offer clinicians and patients a small, low cost, lightweight, easy-to-use alternative for parenteral delivery of liquid medicines.

In response, the applicant of the present application provided a small, low cost, light-weight, easy-to-use device for delivering liquid medicines to a patient. The device, which is described in detail in co-pending U.S. application Ser. No. 09/943,992, filed on Aug. 31, 2001, includes an exit port, a dispenser for causing fluid from a reservoir to flow to the exit port, a local processor programmed to cause a flow of fluid to the exit port based on flow instructions from a separate, remote control device, and a wireless receiver connected to the local processor for receiving the flow instructions. To reduce the size, complexity and costs of the device, the device is provided with a housing that is free of user input components, such as a keypad, for providing flow instructions to the local processor.

What is still desired, however, are new and improved adhesive attachment apparatuses and methods for devices for delivering fluid to a patient. Preferably, the adhesive attachment apparatuses and methods will comfortably and reliably adhere a rigid device, such as a fluid delivery device disclosed in the above-referenced co-pending patent application, to the skin of a patient for extended periods of time for use in effective therapy and diagnosis.

### SUMMARY OF THE INVENTION

The present invention provides a new and improved adhesive attachment apparatus adapted to be disposed between a rigid or semi-rigid device and human skin for reliably attaching the device to the skin for an extended period of time. The apparatus includes a carrier having a skin-contacting surface, and an opposed device-contacting surface, and at least one skin adhesive layer secured to the skin-contacting surface of the carrier for securing the carrier to the skin. A surface area of the skin adhesive layer is less than an area of the skin-contacting surface.

Among other features and benefits, the adhesive attachment apparatus of the present invention has been found to comfortably and reliably adhere a rigid device, such as an insulin infusion device for example, to the skin of a patient for extended periods of time for use in effective insulin therapy.

According to one aspect of the present invention, the carrier includes a passage area for passage of a portion of a device attached by the apparatus between the device-contacting surface and the skin-contacting surface. For example, the passage area allows a needle or transcutaneous cannula of a device to pass through the carrier and enter the skin of a person wearing the apparatus and the device. The skin adhesive layer then preferably includes a layer of adhesive positioned around the passage area of the carrier. According to another aspect, the skin adhesive layer includes a continuous layer of adhesive positioned around the passage area of the carrier.

According to an additional aspect of the present invention, the skin adhesive layer includes a continuous layer of adhesive positioned adjacent an outer edge of the carrier. According to one aspect, the carrier is adapted to extend beyond a perimeter of a bottom external surface of a device attached by the apparatus. According to another aspect, the carrier is adapted to substantially match a perimeter of a bottom external surface of a device attached by the apparatus.

According to one aspect of the present invention, the skin adhesive layer includes separate zones of different properties. According to another aspect, the separate zones of the skin adhesive layer include at least a zone of relative weak adhesive and a zone of relatively strong adhesive. According to an additional aspect, the carrier includes a passage area for passage of a portion of a device attached by the apparatus between the device-contacting surface and the skin-contacting surface, and the zone of relatively strong adhesive is located closer to the passage area than the zone of relative weak adhesive. According to a further aspect, the separate zones of the skin adhesive layer include at least one zone of water-resistant adhesive.

According to one aspect, the separate zones of the skin adhesive layer include at least a zone of relative thin adhesive and a zone of relatively thick adhesive. According to another aspect, the carrier includes a passage area for passage of a portion of a device attached by the apparatus between the device-contacting surface and the skin-contacting surface, and the zone of relatively thick adhesive is located closer to the passage area than the zone of relative thin adhesive.

According to one aspect of the present invention, the skin adhesive layer is annular. According to another aspect, the skin adhesive layer includes an annular array of discontinuous segments.

According to a further aspect of the present invention, the carrier is comprised of resiliently flexible material. According to another aspect, the carrier is comprised of a foam plastic. According to an additional aspect, the carrier is comprised of a closed cell foam plastic.

According to one aspect of the present invention, the carrier is impregnated with an agent. According to another aspect, the agent includes a disinfectant. According to an additional aspect, the agent includes a skin moisturizer. According to yet another aspect, the agent includes an anti-inflammatory. According to a further aspect, the agent includes an antibacterial solution.

According to another aspect of the present invention, the carrier includes a transdermal drug delivery device. According to an additional aspect, the drug delivery device contains a therapeutic diabetes agent.

According to one aspect of the present invention, the carrier includes user-removable portions. According to another aspect, the user-removable portions of the carrier include successive outer peripheral rings of the carrier.

According to one aspect of the present invention, the carrier includes separate zones of different properties. The separate zones of the carrier include a zone of relatively thick material and a zone of relatively thin material. According to an additional aspect, the separate zones of the carrier include a zone of relatively porous material and a zone of relatively non-porous material. According to a further aspect, the separate zones of the carrier include a first zone peripherally surrounded by a second zone. According to yet an additional aspect, the second zone of the carrier has a thickness that decreases further from the first zone of the carrier. According to still another aspect, the separate zones of the carrier include first and second spaced-apart zones peripherally surrounded by a third zone. According to a further aspect, the third zone of the carrier has a thickness that decreases further from the first and the second zones of the carrier.

According to still another aspect of the present invention, the carrier includes a material that physically changes upon contacting a predetermined fluid. According to an additional aspect, the predetermined fluid includes insulin. According to a further aspect, the physical change includes a change in color. According to another aspect, the physical change includes a change in thickness. According to a further aspect, the physical change includes a change in electrical conductivity.

According to yet another aspect of the present invention, the skin adhesive layer includes a material that physically changes upon contacting a predetermined fluid. According to one aspect, the predetermined fluid includes insulin. According to an additional aspect, the physical change includes a change in color.

According to a further aspect of the present invention, the skin adhesive layer includes user-removable portions. According to one aspect, the user-removable portions of the skin adhesive layer include successive outer peripheral rings of the skin adhesive layer.

According to an additional aspect of the present invention, the carrier includes a tab extending therefrom, and the skin adhesive layer includes discontinuities extending towards the tab.

According to a further aspect of the present invention, the carrier includes a plurality of removable stacked layers, and each layer is separated by an intermediate adhesive layer. According to one aspect, surface areas of the intermediate adhesive layers are less than areas of surfaces of the stacked layers. According to another aspect, the layers of the carrier are provided in different colors. According to an additional aspect, the layers of the carrier are labeled.

According to one aspect of the present invention, an antenna is embedded in the carrier and includes at least one end extending from the device-contacting surface. According to another aspect, a conductor is embedded in the carrier and includes opposing ends extending from the device-contacting surface.

According to another aspect, at least one sensor is embedded in the carrier. The sensor is adapted to provide an indication of whether the skin-contacting surface of the carrier remains in contact with a patient's skin upon the apparatus being attached to a patient's skin. According to one aspect, the sensor includes a temperature sensor. According to another aspect, the sensor includes an impedance sensor.

According to one aspect of the present invention, an attachment mechanism is secured to the carrier. According to another aspect, the attachment mechanism includes a clip. According to a further aspect of the present invention, a label is secured to the carrier.

According to yet another aspect of the present invention, the carrier includes first and second layers separated by a bladder. According to one aspect, the bladder contains a gel.

According to still another aspect of the present invention, the carrier includes at least two layers connected by resiliently flexible spacers.

According to a further aspect of the present invention, the means for securing the device-contacting surface of the carrier against a bottom external surface of a device attached by the apparatus includes at least one device adhesive layer secured to the device-contacting surface of the carrier. According to one aspect, a surface area of the device adhesive layer is less than an area of the device-contacting surface. According to another aspect, the carrier includes a passage area for passage of a portion of a device attached by the apparatus between the device-contacting surface and the skin-contacting surface, and the device adhesive layer includes a first annular layer of adhesive secured to the device-contacting surface of the carrier and positioned around the passage area of the carrier. According to a further aspect, the device adhesive layer also includes a second layer of adhesive spaced from the first layer. According to another aspect, the device adhesive layer includes first and second layers separated by a bladder.

According to still another aspect of the present invention, the means for securing the device-contacting surface of the carrier against a bottom external surface of a device includes a strap connected to the carrier for passing around the device and holding the device against the device-contacting surface of the carrier. According to one aspect, the strap extends between ends of the carrier. According to an additional aspect, means for securing further includes a layer of adhesive secured to the strap for securing the strap to a device attached by the apparatus.

According to one aspect of the present invention, the means for securing the device-contacting surface of the carrier against a bottom external surface of a device attached by the apparatus includes at least one pocket secured to the carrier for receiving the device therein and holding the device against the device-contacting surface of the carrier. According to another aspect, the pocket is positioned at an end of the carrier. According to a further aspect, the apparatus includes two of the pockets positioned at opposite ends of the carrier.

According to one aspect of the present invention, the means for securing the device-contacting surface of the carrier against a bottom external surface of a device attached by the apparatus includes a mechanical connector. According to another aspect, the mechanical connector includes at least one socket for receiving a stud of the device in a snap-fit manner. According to a further aspect, the mechanical connector includes at least one stud for being received in a socket of the device in a snap-fit manner.

According to one aspect of the present invention, the carrier includes at least two layers connected by a mechanical connector. According to another aspect, the mechanical connector includes a socket secured to one of the layers of the carrier receiving a stud secured to the other of the layers of the carrier in a snap-fit manner.

According to one aspect of the present invention, the means for securing the device-contacting surface of the carrier against a bottom external surface of a device attached by the apparatus includes at least one intermediate layer of flexible material secured to the device-contacting surface of the carrier, and at least one device adhesive layer secured to the intermediate layer of flexible material for adhesion against a bottom external surface of a device attached by the apparatus. According to another aspect, the intermediate layer of flexible material is secured to the device-contacting surface of the carrier by an intermediate layer of adhesive. According to an additional aspect, the intermediate layer of flexible material includes two spaced-apart intermediate layers of flexible material.

According to one aspect of the present invention, the apparatus further includes a compartment positioned adjacent the skin contacting surface of the carrier and containing adhesive remover, and a release mechanism extending from the compartment for opening the compartment.

The present invention also provides a device for delivering fluid to a patient, including an exit port assembly adapted to connect to a transcutaneous patient access tool, a dispenser for causing fluid from a reservoir to flow to the exit port assembly, a local processor connected to the dispenser and programmed to cause a flow of fluid to the exit port assembly based on flow instructions, a wireless receiver connected to the local processor for receiving flow instructions from a separate, remote control device and delivering the flow instructions to the local processor, and a housing containing the exit port assembly, the dispenser, the local processor, and the wireless receiver, and having an external surface. The housing also includes feet extending from the external surface to distal surfaces, and separate skin adhesive layers are secured to the distal surfaces of each foot for securing the device to a skin surface of a patient.

According to one aspect of the present invention, surface areas of the skin adhesive layers are less than areas of the distal surfaces of the feet of the housing. According to another aspect, surface areas of the skin adhesive layers are about equal to areas of the distal surfaces of the feet of the housing.

The present invention also provides a device for delivering fluid to a patient, including an exit port assembly adapted to connect to a transcutaneous patient access tool, a dispenser for causing fluid from a reservoir to flow to the exit port assembly, a local processor connected to the dispenser and programmed to cause a flow of fluid to the exit port assembly based on flow instructions, a wireless receiver connected to the local processor for receiving flow instructions from a separate, remote control device and delivering the flow instructions to the local processor, and a housing containing the exit port assembly, the dispenser, the local processor, and the wireless receiver, and having an external surface. The housing also has legs extending from the external surface to distal overhanging feet, and a carrier having a skin-contacting surface and an opposed device-contacting surface, is positioned between the external surface of the housing and the overhanging feet and has openings receiving the legs. At least one skin adhesive layer secured to the skin-contacting surface of the carrier.

These aspects of the invention together with additional features and advantages thereof may best be understood by reference to the following detailed descriptions and examples taken in connection with the accompanying illustrated drawings.

### BRIEF DESCRIPTION OF THE DRAWINGS

FIG. 1 is a perspective view of an exemplary embodiment of a fluid delivery device constructed in accordance with the present invention shown secured on a patient with an exemplary embodiment of a skin attachment apparatus constructed in accordance with the present invention, and a remote control device for use with the fluid delivery device (the remote control device being enlarged with respect to the patient and the fluid delivery device for purposes of illustration);

FIG. 2 is an enlarged side elevation view of the fluid delivery device and the skin attachment apparatus of FIG. 1;

FIG. 3 is an enlarged top plan view of the fluid delivery device and the skin attachment apparatus of FIG. 1;

FIG. 4 is an enlarged top plan view of the skin attachment apparatus of FIG. 1;

FIG. 5 is an enlarged bottom plan view of the skin attachment apparatus of FIG. 1;

FIG. 6 is an enlarged side elevation view of the fluid delivery device of FIG. 1 and another exemplary embodiment of a skin attachment apparatus constructed in accordance with the present invention;

FIG. 7 is an enlarged top plan view of the fluid delivery device and the skin attachment apparatus of FIG. 6;

FIG. 8 is an enlarged top plan view of the skin attachment apparatus of FIG. 6;

FIG. 9 is an enlarged bottom plan view of the skin attachment apparatus of FIG. 6;

FIG. 10 is a bottom plan view of an additional exemplary embodiment of a skin attachment apparatus constructed in accordance with the present invention;

FIG. 11 is a bottom plan view of an exemplary embodiment of an adhesive layer constructed in accordance with the present invention;

FIG. 12 is a bottom plan view of another exemplary embodiment of an adhesive layer constructed in accordance with the present invention;

FIG. 13 is a bottom plan view of an additional exemplary embodiment of an adhesive layer constructed in accordance with the present invention;

FIG. 14 is a side elevation view of a fluid delivery device and an exemplary embodiment of a skin attachment apparatus constructed in accordance with the present invention;

FIG. 15 is a side elevation view of a fluid delivery device and another exemplary embodiment of a skin attachment apparatus constructed in accordance with the present invention;

FIG. 16 is a bottom plan view of another exemplary embodiment of an adhesive layer constructed in accordance with the present invention;

FIG. 17 is a top plan view of another skin attachment apparatus constructed in accordance with the present invention;

FIG. 18 is a side elevation view of the skin attachment apparatus of FIG. 17;

FIG. 19 is a top plan view of an additional skin attachment apparatus constructed in accordance with the present invention;

FIG. 20 is a top plan view of a fluid delivery device and another exemplary embodiment of a skin attachment apparatus constructed in accordance with the present invention;

FIG. 21 is a side elevation view of the fluid delivery device and the skin attachment apparatus of FIG. 20;

FIG. 22 is a top plan view of a fluid delivery device and an additional exemplary embodiment of a skin attachment apparatus constructed in accordance with the present invention;

FIG. 23 is a side elevation view of the fluid delivery device and the skin attachment apparatus of FIG. 22;

FIG. 24 is a side elevation view, partially cut-away, of yet other exemplary embodiments of a fluid delivery device and a skin attachment apparatus constructed in accordance with the present invention;

FIG. 25 is a top plan view of the fluid delivery device of FIG. 24;

FIG. 26 is a bottom plan view of the skin attachment apparatus of FIG. 24;

FIG. 27 is a bottom plan view of the skin attachment apparatus of FIG. 24, shown after contacting fluid leaked from the fluid delivery device but not delivered to a patient;

FIG. 28 is a bottom plan view of a further exemplary embodiment of a skin attachment apparatus constructed in accordance with the present invention;

FIG. 29 is a side elevation view of exemplary embodiments of a fluid delivery device and a skin attachment apparatus constructed in accordance with the present invention;

FIG. 30 is a bottom plan view of another exemplary embodiment of a skin attachment apparatus constructed in accordance with the present invention;

FIG. 31 is a bottom plan view of an additional exemplary embodiment of a skin attachment apparatus constructed in accordance with the present invention;

FIG. 32 is a side elevation view of other exemplary embodiments of a fluid delivery device and a skin attachment apparatus constructed in accordance with the present invention;

FIG. 33 is an exploded side elevation view of yet other exemplary embodiments of a fluid delivery device and a skin attachment apparatus constructed in accordance with the present invention;

FIG. 34 is an exploded side perspective view of still other exemplary embodiments of a fluid delivery device and a skin attachment apparatus constructed in accordance with the present invention;

FIG. 35 is a bottom plan view of an exemplary embodiment of a skin attachment apparatus constructed in accordance with the present invention;

FIG. 36 is an enlarged sectional view of the skin attachment apparatus taken along line 36-36 of FIG. 35;

FIG. 37 is a side elevation view of exemplary embodiments of a fluid delivery device and a skin attachment apparatus constructed in accordance with the present invention;

FIG. 38 is an enlarged sectional view of portions of the fluid delivery device and the skin attachment apparatus of FIG. 37;

FIG. 39 is a bottom plan view of an exemplary embodiment of a fluid delivery device constructed in accordance with the present invention;

FIG. 40 is a side elevation view, partially cut-away, of the fluid delivery device of FIG. 39;

FIG. 41 is an exploded top perspective view of further exemplary embodiments of a fluid delivery device and a skin attachment apparatus constructed in accordance with the present invention;

FIG. 42 is a side elevation view of yet other exemplary embodiments of a fluid delivery device and a skin attachment apparatus constructed in accordance with the present invention;

FIG. 43 is a top plan view of additional exemplary embodiments of a fluid delivery device and a skin attachment apparatus constructed in accordance with the present invention;

FIG. 44 is a top plan view of exemplary embodiments of a fluid delivery device and a skin attachment apparatus constructed in accordance with the present invention;

FIG. 45 is a side elevation view of still other exemplary embodiments of a fluid delivery device and a skin attachment apparatus constructed in accordance with the present invention;

FIG. 46 is a top plan view of the fluid delivery device and the skin attachment apparatus of FIG. 45;

FIG. 47 is an exploded side elevation view of more exemplary embodiments of a fluid delivery device and a skin attachment apparatus constructed in accordance with the present invention;

FIG. 48 is a side elevation view of other exemplary embodiments of a fluid delivery device and a skin attachment apparatus constructed in accordance with the present invention;

FIG. 49 is a side elevation view of another exemplary embodiment of a fluid delivery device constructed in accordance with the present invention;

FIG. 50 is a side elevation view of an additional exemplary embodiment of a fluid delivery device constructed in accordance with the present invention;

FIG. 51 is a bottom plan view of a further exemplary embodiment of a fluid delivery device constructed in accordance with the present invention; and

FIG. 52 is a side elevation view of other exemplary embodiments of a fluid delivery device and a skin attachment apparatus constructed in accordance with the present invention.

Like reference characters designate identical or corresponding components and units throughout the several views.

### DETAILED DESCRIPTION OF THE EXEMPLARY EMBODIMENTS

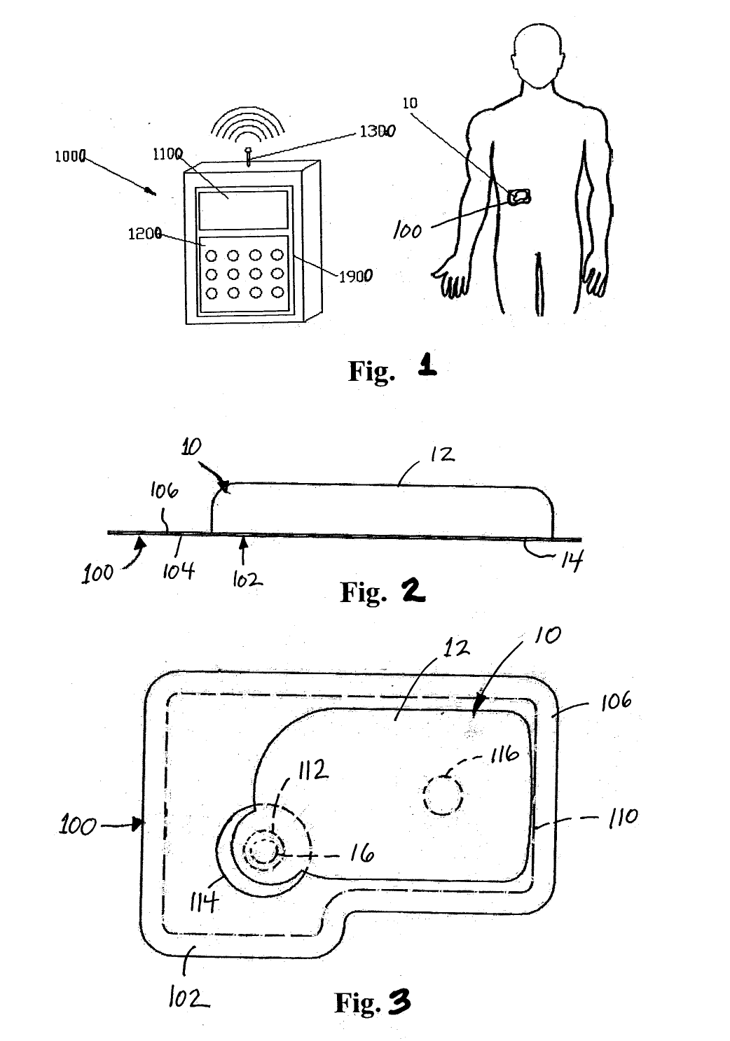

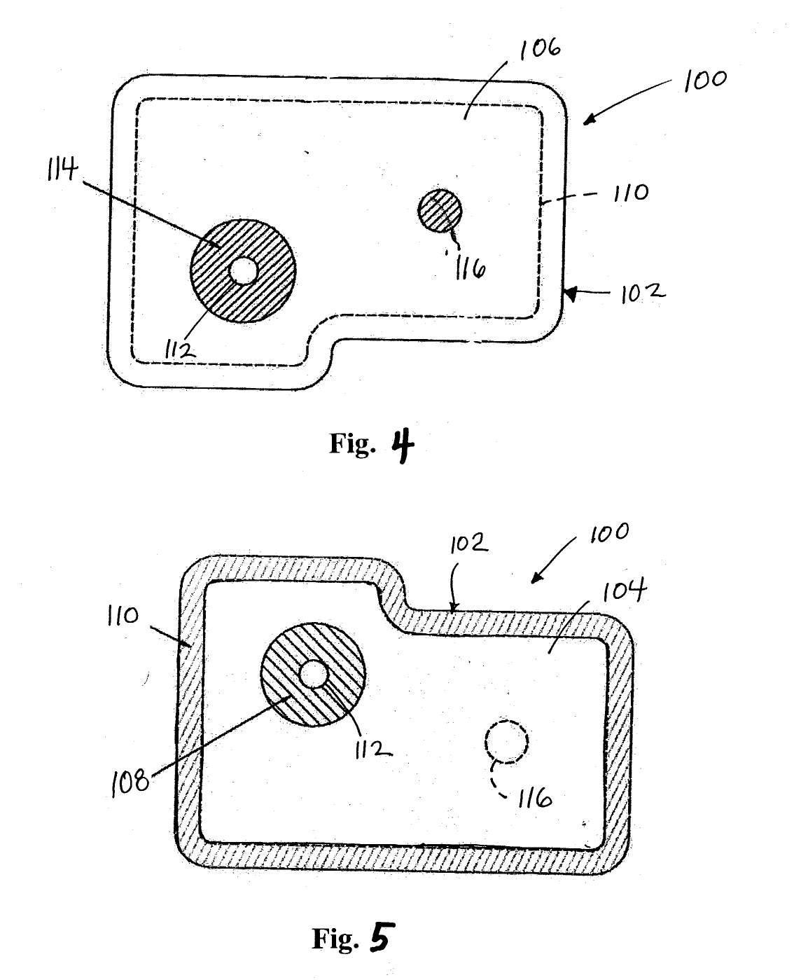

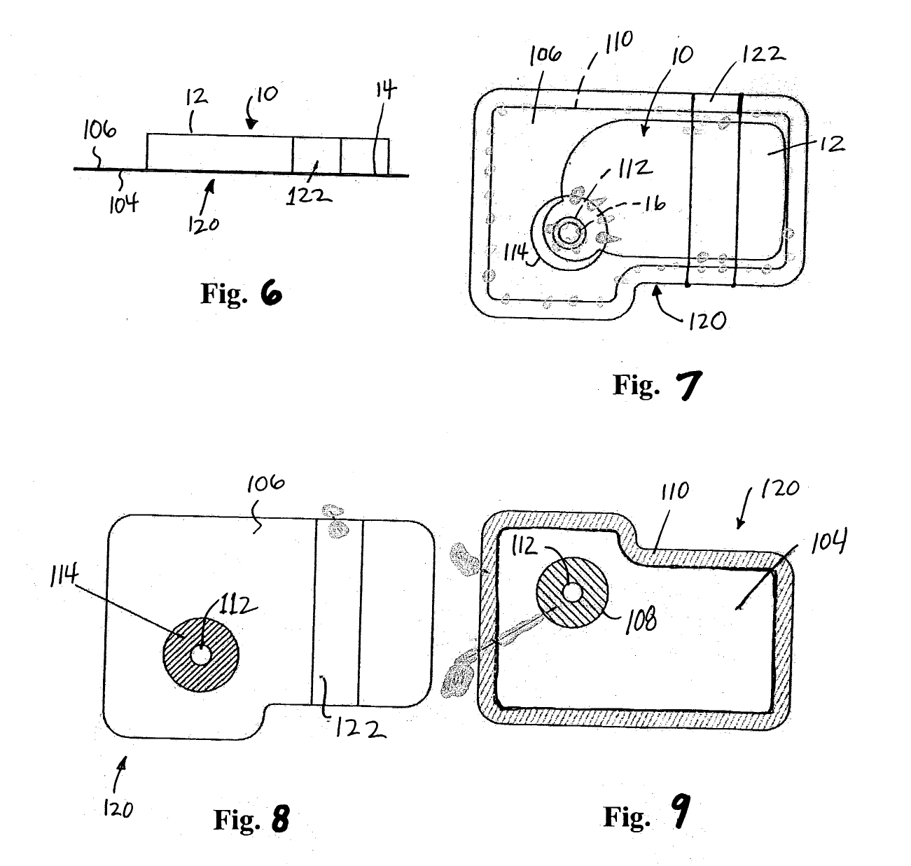

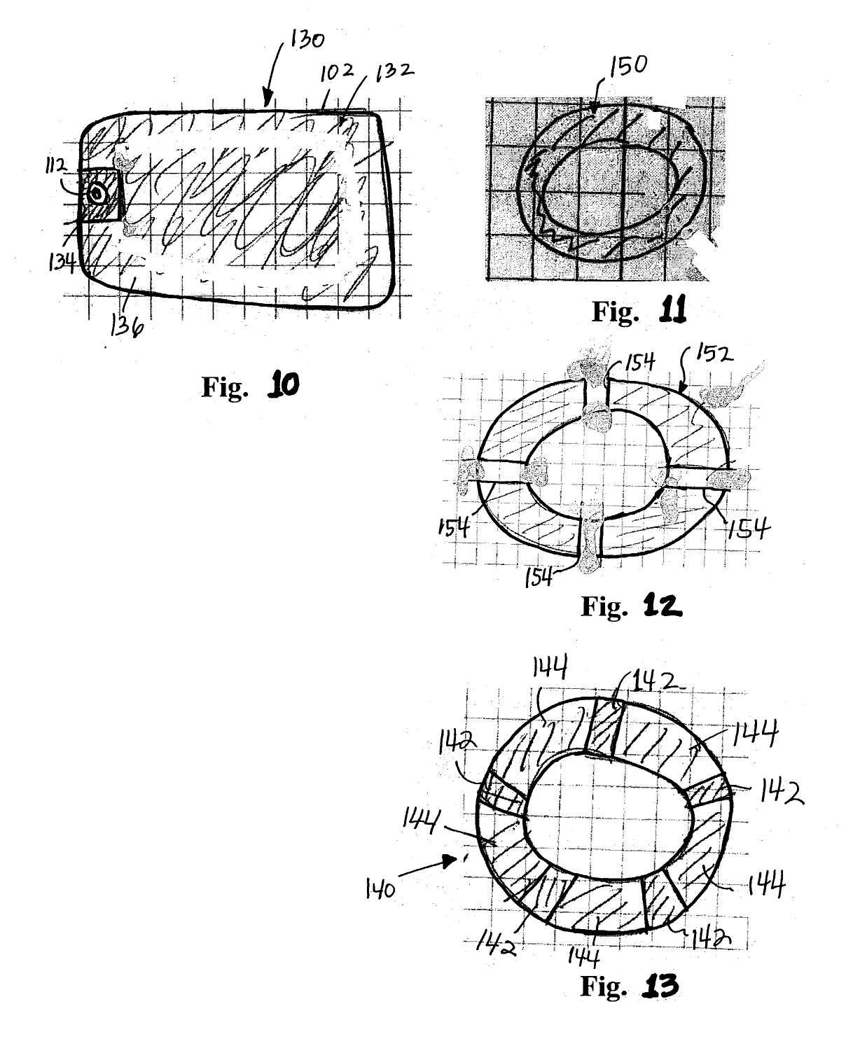

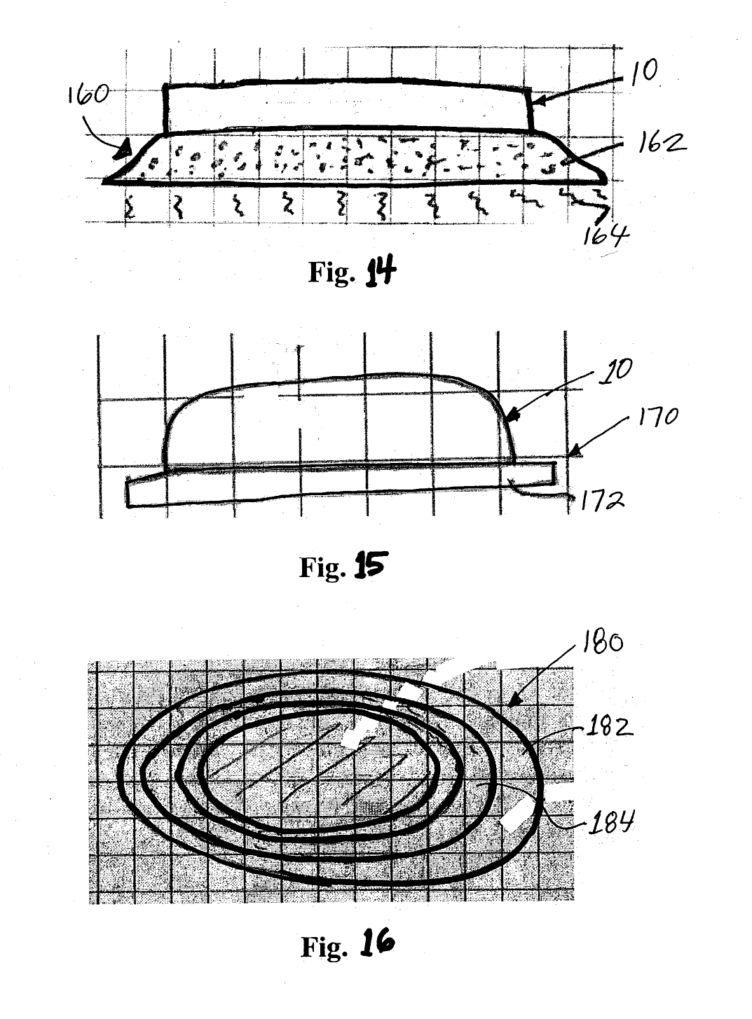

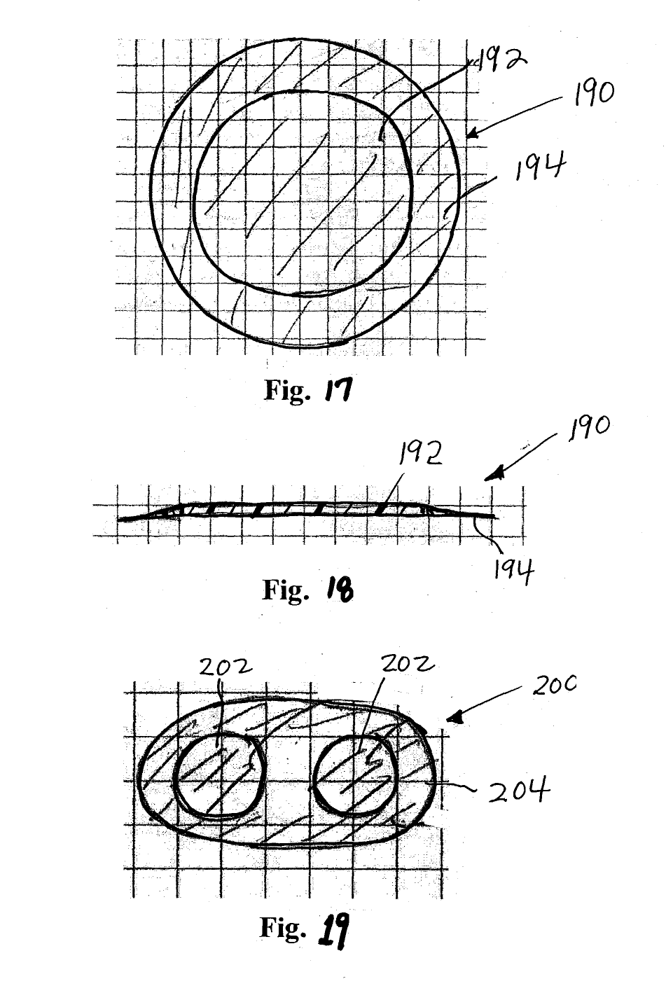

Referring to FIGS. 1 through 3, there is illustrated an exemplary embodiment of a fluid delivery device 10 constructed in accordance with the present invention, which can be used for the delivery of fluids to a person or animal. The fluid delivery device is provided with an exemplary embodiment of new and improved adhesive attachment apparatus 100 constructed in accordance with the present invention and adapted to be disposed between the fluid delivery device 10 and human skin for comfortably and reliably attaching the device 10 to the skin of a patient, as illustrated in FIG. 1, for an extended period of time.

Before the adhesive attachment apparatus 100 is discussed in further detail, however, the fluid delivery device 10 will first be described to provide some background information. The types of liquids that can be delivered by the fluid delivery device 10 include, but are not limited to, insulin, antibiotics, nutritional fluids, total parenteral nutrition or TPN, analgesics, morphine, hormones or hormonal drugs, gene therapy drugs, anticoagulants, analgesics, cardiovascular medications, AZT or chemotherapeutics. The types of medical conditions that the fluid delivery device 10 might be used to treat include, but are not limited to, diabetes, cardiovascular disease, pain, chronic pain, cancer, AIDS, neurological diseases, Alzheimer's Disease, ALS, Hepatitis, Parkinson's Disease or spasticity. The volume of an internal reservoir of the fluid delivery device 10 is chosen to best suit the therapeutic application of the fluid delivery device 10 impacted by such factors as available concentrations of medicinal fluids to be delivered, acceptable times between refills or disposal of the fluid delivery device 10, size constraints and other factors.

The fluid delivery device 10 also includes a dispenser for causing fluid from the reservoir to flow to a transcutaneous access tool, such as a skin penetrating cannula. A processor or electronic microcontroller (hereinafter referred to as the “local” processor) is connected to the dispenser, and is programmed to cause a flow of fluid to the transcutaneous access tool based on flow instructions from a separate, remote control device 1000, an example of which is shown in FIG. 1. A wireless receiver is connected to the local processor for receiving flow instructions from the remote control device 1000 and delivering the flow instructions to the local processor. The device 10 also includes an external housing 12 containing the transcutaneous access tool, the reservoir, the dispenser, the local processor, and the wireless receiver.

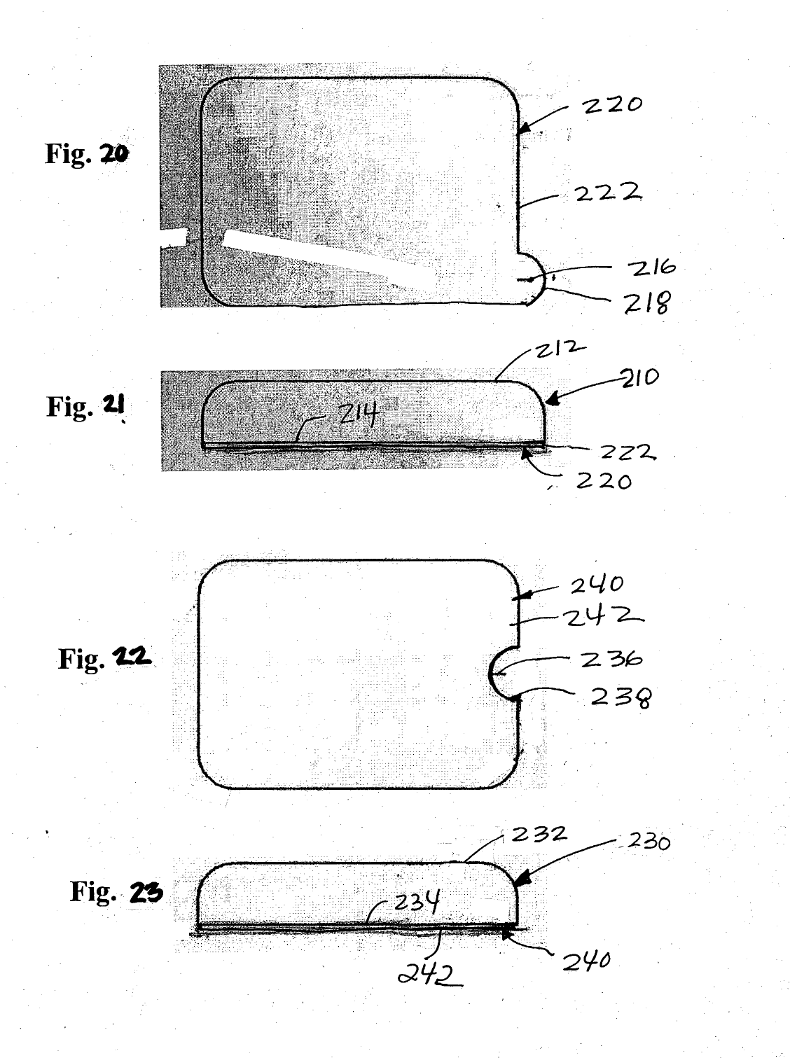

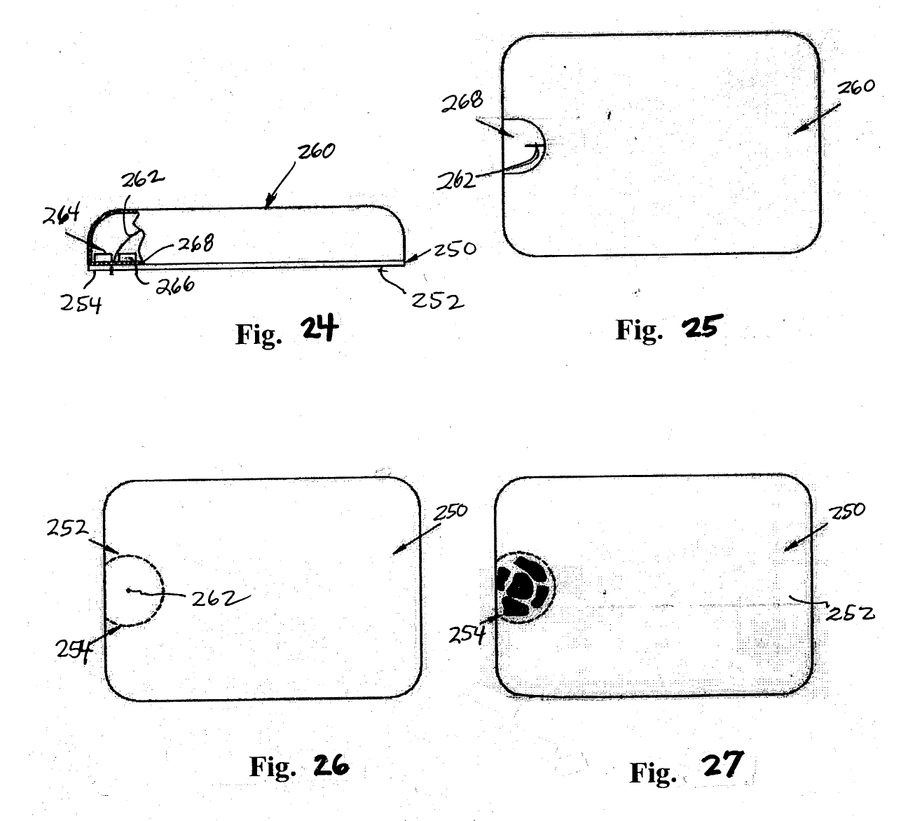

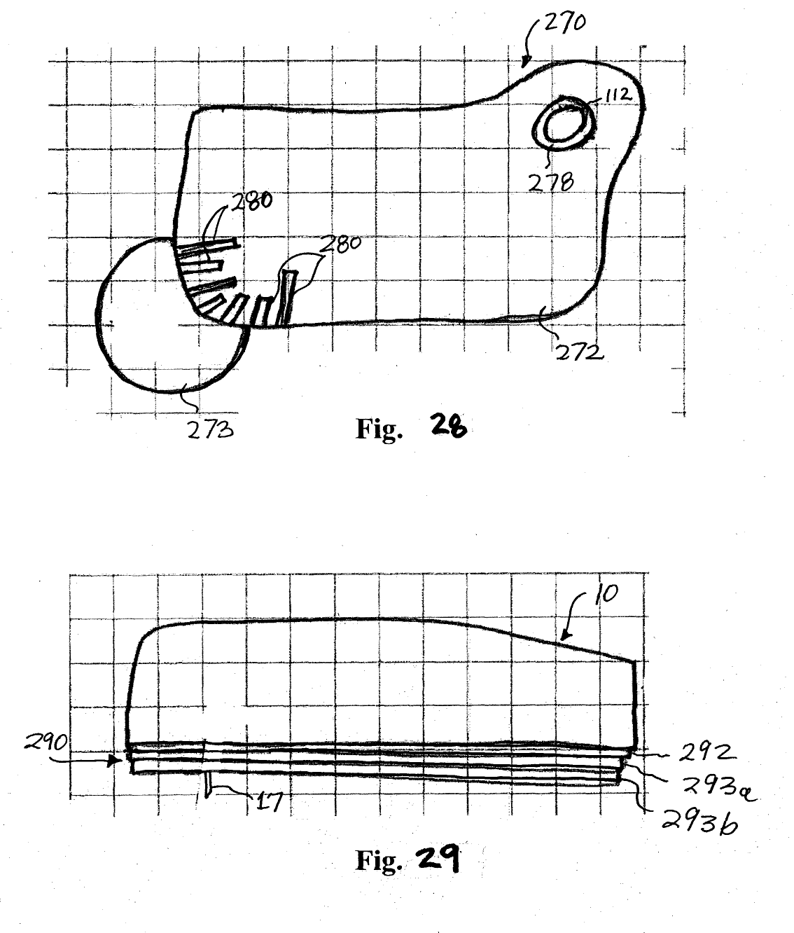

As shown best in FIGS. 2 and 3, the housing 12 of the fluid delivery device 10 is free of user input components for providing flow instructions to the local processor, such as electromechanical switches or buttons on an outer surface of the housing 12, or interfaces otherwise accessible to a user to adjust the programmed flow rate through the local processor. The lack of user input components allows the size, complexity and costs of the device 10 to be substantially reduced so that the device 10 lends itself to being small and disposable in nature. Examples of such devices are disclosed in co-pending U.S. patent application Ser. No. 09/943,992, filed on Aug. 31, 2001 (Atty. Docket No. INSL-110), and entitled DEVICES, SYSTEMS AND METHODS FOR PATIENT INFUSION, which is assigned to the assignee of the present application and has previously been incorporated herein by reference.

In the exemplary embodiment of FIGS. 2 and 3, the housing 12 is relatively rigid. However, the device 10 can be provided with a flexible housing and/or a housing having hinges or bendable portions for allowing the housing to bend, in order to accommodate patient movement and comfort.

In order to program, adjust the programming of, or otherwise communicate user inputs to the local processor, the fluid delivery device 10 includes the wireless communication element, or receiver, for receiving the user inputs from the separate, remote control device 1000 of FIG. 1. Signals can be sent via a communication element (not shown) of the remote control device 1000, which can include or be connected to an antenna 1300, shown in FIG. 1 as being external to the device 1000.

The remote control device 1000 has user input components, including an array of electromechanical switches, such as the membrane keypad 1200 shown. The remote control device 1000 also includes user output components, including a visual display, such as a liquid crystal display (LCD) 1100. Alternatively, the control device 1000 can be provided with a touch screen for both user input and output. Although not shown in FIG. 1, the remote control device 1000 has its own processor (hereinafter referred to as the “remote” processor) connected to the membrane keypad 1200 and the LCD 1100. The remote processor receives the user inputs from the membrane keypad 1200 and provides “flow” instructions for transmission to the fluid delivery device 10, and provides information to the LCD 1100. Since the remote control device 1000 also includes a visual display 1100, the fluid delivery device 10 can be void of an information screen, further reducing the size, complexity and costs of the device 10.

The device 10 preferably receives electronic communication from the remote control device 1000 using radio frequency or other wireless communication standards and protocols. In a preferred embodiment, the communication element of the device 10 is a two-way communication element, including a receiver and a transmitter, for allowing the fluid delivery device 10 to send information back to the remote control device 1000. In such an embodiment, the remote control device 1000 also includes an integral communication element comprising a receiver and a transmitter, for allowing the remote control device 1000 to receive the information sent by the fluid delivery device 10.

The local processor of the device 10 contains all the computer programs and electronic circuitry needed to allow a user to program the desired flow patterns and adjust the program as necessary. Such circuitry can include one or more microprocessors, digital and analog integrated circuits, resistors, capacitors, transistors and other semiconductors and other electronic components known to those skilled in the art. The local processor also includes programming, electronic circuitry and memory to properly activate the dispenser at the needed time intervals.

Referring to FIGS. 2 and 3, the exemplary embodiment of the adhesive attachment apparatus 100 of the present invention is provided on an external “bottom” surface 14 of the housing 12, which defines an external exit port 16 of the housing 12. The external exit port 16 allows the transcutaneous access tool (e.g., a rigid needle or a soft cannula) to extend out of the device 10 and into the skin of a patient wearing the device 10.

The apparatus 100 is also shown in FIGS. 4 and 5. In general, the adhesive attachment apparatus 100 includes a carrier 102 having a skin-contacting surface 104, and an opposed device-contacting surface 106. The adhesive attachment apparatus 100 also includes at least one skin adhesive layer 108, 110 (skin adhesive layer 108 is viewable in FIG. 5) secured to the skin-contacting surface 104 of the carrier 102 for securing the carrier to the skin. The skin adhesive layers 108, 110 are adapted such that a total surface area of the skin adhesive layers 108, 110 are less than an area of the skin-contacting surface 104. Reduced adhesive area has numerous advantages including, but not limited to, less adhesive coverage of the patient's skin and easier removal.

Among other features and benefits, the adhesive attachment apparatus 100 of the present invention has been found to comfortably and reliably adhere a rigid device, such as the fluid device 10 of FIGS. 1 through 3 for example, to the skin of a patient for extended periods of time for use in effective therapy and/or diagnostics. In an exemplary embodiment, the adhesive attachment apparatus 100 is designed to comfortably and reliably adhere the fluid device 10 of FIGS. 1 through 3 for example, to the skin of a patient for up to three days (72 hours) in order to continuously delivery insulin to the patient for the treatment of diabetes.

It should be understood however, that the adhesive attachment apparatus 100 of the present invention can be used with devices other than the exemplary embodiment of the device 10 of FIGS. 1 through 3. For example, the adhesive attachment apparatus 100 of the present invention can be used with other types of medical treatment devices and medical diagnostic devices (e.g., a blood glucose or other physiologic parameter monitoring device), and can also be used with non-medical devices, that are desired to be comfortably and reliably adhered to the skin of a patient for extended periods of time.

In the exemplary embodiment shown in FIGS. 3 through 5, the carrier 102 includes a passage area 112 that extends between the device-contacting surface 106 and the skin-contacting surface 104 for passage of a portion of the device 10 attached by the apparatus 100. For example, the passage area 112 can comprise an opening or a weakened area of the carrier 102 for allowing the transcutaneous access tool (e.g., a soft cannula) to extend out of the external exit port 16 of the device 10 and into the skin of a patient wearing the device 10. In the exemplary embodiment shown, the passage area comprises a circular opening 112.

A first 108 of the skin adhesive layers is positioned around the passage area 112 of the carrier 102. As shown, the first skin adhesive layer 108 is annular and continuously extends around the passage area of the carrier 102. During flexing of the carrier 102, when attached to the skin surface, the first skin adhesive layer 108 surrounding the passage area 112 of the carrier 102 allows portions of the carrier 102 remote from the passage area 112 to pivot with respect to the skin surface about the first skin adhesive layer 108. However, the first skin adhesive layer 108 surrounding the passage area 112 secures the portions of the carrier 102 directly adjacent to the passage area 112 to the skin surface. In this manner, the first skin adhesive layer 108 allows the carrier 102 to flex for patient comfort, yet prevents bending and kinking of the transcutaneous access tool extending through the passage area 112.

In the exemplary embodiment of FIGS. 3 through 5, a second 110 of the skin adhesive layers comprises a continuous layer of adhesive positioned adjacent an outer edge of the carrier 102., According to one exemplary embodiment of the present invention, the skin adhesive layers 108, 110 comprise pressure sensitive adhesives. Such pressure sensitive adhesives include acrylic, butyl, hydrogel, polyisobutylene, silicone and the like adhesives. The thickness of the adhesives 108, 110 are about between 1.5 and 2.0 mils. The skin adhesive layers 108, 110 can comprise an adhesive transfer tape system, such as Fastape™ from Avery Dennison. Although not shown, the skin adhesive layers 108, 110 are covered with a conventional release liner prior to use. Standard release liners made of low cost siliconised paper or plastic film can be employed.

As shown in FIGS. 1 through 3, the carrier 102 is sized and adapted to extend beyond an outer perimeter of the bottom external surface 14 of the fluid delivery device 10 attached by the apparatus. The “oversized” carrier 102 has been found to provide additional stability in some cases. Alternatively, the carrier 102 may be sized to match the perimeter of the fluid delivery device 10, have a boundary that ends under the perimeter of the fluid delivery device 10, or that alternatively has a circuitous boundary that in some locations extends beyond and in other locations ends under the perimeter of the fluid delivery device 10.

The carrier 102 preferably has viscoelastic properties approaching that of skin or in some cases more flexible than skin. The carrier 0.102 can be formed from foams, especially flexible foams manufactured by Kendall Polychem or and Avery Dennison. In particular, the preferred foams include Actiflex, made by Kendall Polychem, and a PVC closed cell foam made by Avery Dennison under the model number Q527297. 3M 9773 foam tape can also be used for the carrier 102. The carrier 102 can also be made of woven and non-woven fabrics. A suitable non-woven fabric is a spun-laced polyester marketed by Du Pont under the mark Sontara™. Tagaderm™ transparent film from 3M can also be used for the carrier 102.

The adhesive attachment apparatus 100 also includes means for securing the device-contacting surface 106 of the carrier 102 against the bottom external surface 14 of the device 10. In the exemplary embodiment of FIGS. 1 through 3, the means for securing the device-contacting surface 106 of the carrier 102 against a bottom external surface of the device 10 includes at least one device adhesive layer 114, 116 secured to the device-contacting surface 106 of the carrier 102. As shown in FIGS. 3 through 5, the device adhesive layers 114, 116 are adapted such that a total surface area of the device adhesive layers 114, 116 are less than an area of the device-contacting surface 106.

As also shown in FIGS. 3 through 5, a first 114 of the device adhesive layers is annular in shape and is secured to the device-contacting surface 106 of the carrier 102 around the passage area 112 of the carrier 102. A second 116 of the device adhesive layers is circular in shape and spaced from the first device adhesive layer 114.

According to one exemplary embodiment of the present invention, the device adhesive layers 114, 116 comprise pressure sensitive adhesives, similar to the skin adhesive layers 108, 110 as discussed above. The device adhesive layers 114, 116 can be covered with a conventional release liner prior to use, if the apparatus 100 is provided to a user separate from the fluid delivery device 10.

Referring now to FIGS. 6 through 9, another exemplary embodiment of an adhesive attachment apparatus 120 constructed in accordance with the present invention is shown. The apparatus 120 is similar to the apparatus 100 of FIGS. 1 through 5, such that similar elements have the same reference numerals. In the apparatus 120 of FIGS. 6 through 9, however, the means for securing the device-contacting surface 106 of the carrier 102 against the bottom external surface 14 of the device 10 includes the annular device adhesive layer 114 positioned around the passage area 112 of the carrier 102, and a strap 122 spaced from the device adhesive layer 114. The strap 122 is connected to the carrier 102 and passes around the device 10 and holds the device against the device-contacting surface 106 of the carrier. Preferably the strap 122 is made of a flexibly resilient material to allow the device to move with respect to the carrier 102. The device 10, however, is prevented by the annular device adhesive layer 114 from moving with respect to the carrier 102 directly around the passage area 112 of the carrier. In effect, therefore, the device 10 can pivot about the passage area 112 of the carrier 102. Ends of the strap 122 are secured to the carrier in a suitable manner, such as by gluing or bonding.

Referring to FIGS. 10 and 13, the skin adhesive layer(s) of an adhesive attachment apparatus constructed in accordance with the present invention can be provided with separate zones of different properties. For example, the skin adhesive layer(s) can be provided with separate zones of the same type of adhesive but having different strengths and/or thicknesses. The skin adhesive layer(s) can also be provided with separate zones of different types of adhesive having varied properties such as strength, solubility, permeability and thickness. In the exemplary embodiment of FIG. 10, an adhesive attachment apparatus 130 constructed in accordance with the present invention is provided with a skin adhesive layer 132 having a zone 134 of relatively stronger adhesive and a zone 136 of relatively weaker adhesive. Preferably, the zone 134 of relatively stronger adhesive is positioned around the passage area 112 of the carrier 102. In the exemplary embodiment of FIG. 13, a skin adhesive layer 140 constructed in accordance with the present invention is provided with zones 142 of a first type of adhesive and zones 144 of a second type of adhesive. Preferably, the first type of adhesive 142 is a water-activated adhesive, while the second type 144 of adhesive is a dry adhesive, such that resulting adhesive attachment apparatus works well when dry and when wet (e.g., for when a user wears the apparatus while taking a shower).

Referring to FIGS. 11 and 12, skin adhesive layers 150, 152 of an adhesive attachment apparatus constructed in accordance with the present invention can be provided with annular shapes. The annular skin adhesive layers 150, 152 allow for carrier flexibility within the annular skin adhesive layer. The annular skin adhesive layer 152 of FIG. 12 further includes discontinuities 154 (i.e., annular array of discontinuous segments) that allow air and moisture to flow into and out of the annular layer.

Referring to FIG. 14, another exemplary embodiment of an adhesive attachment apparatus 160 constructed in accordance with the present invention is shown. The apparatus 160 is similar to the apparatus 100 of FIGS. 1 through 5. In the apparatus 160 of FIG. 14, however, a carrier 162 of the apparatus 160 is made of material that is impregnated or coated with a desired agent. The agent can comprise, for example, a disinfectant, a skin moisturizer, or an antibacterial formula. The carrier 162 and the agent can be adapted such that the agent gradually seeps out of the carrier 162 onto the skin of a patient during use of the apparatus 160, as illustrated by lines 164 in FIG. 14.

Referring to FIG. 15, an additional exemplary embodiment of an adhesive attachment apparatus 170 constructed in accordance with the present invention is shown. The apparatus 170 is similar to the apparatus 100 of FIGS. 1 through 5. In the apparatus 170 of FIG. 15, however, a carrier 172 of the apparatus 170 comprises a transdermal drug delivery device containing a therapeutic diabetes agent. In one exemplary embodiment, the transdermal drug delivery device 172 contains a therapeutic diabetes agent. The transdermal delivery device, whose construction is well known to those of skill in the art, enables the patient to receive a medication or other agent transdermally throughout the use of the device attached by attachment apparatus 170. If the device is a drug delivery device, the agent may be a different drug, to allow multiple drug therapy, or the same drug provided in an additional delivery profile. Providing a transdermal delivery device within carrier 172 enables the patient to receive dual therapies or a combined diagnostic function (i.e. the device is a diagnostic device) and transdermal drug delivery therapy with a single assembly, covering a single portion of the patient's skin.

Referring to FIG. 16, a skin adhesive layer 180 of an adhesive attachment apparatus constructed in accordance with the present invention can be provided with user-removable portions 182, 184. In this manner, a user can remove one or more of the portions 182, 184 if he or she feels that a smaller amount of adhesive can adequately secure a device to the user's skin. In the exemplary embodiment of FIG. 16, the user-removable portions 182, 184 of the skin adhesive layer 180 include successive outer peripheral rings 182, 184 of the skin adhesive layer 180. Although not shown, a carrier of an adhesive attachment apparatus constructed in accordance with the present invention can also be provided user-removable portions.

Referring to FIGS. 17 and 18, a carrier 190 of an adhesive attachment apparatus constructed in accordance with the present invention can be provided separate zones 192, 194 of different properties. For example, in the exemplary embodiment of FIGS. 17 and 18, the carrier 190 includes a circular central zone 192 of relatively thicker material, such as foam, and an annular outer zone 194 of relatively thin material, such as a thin film. The circular central zone 192 can be made of relatively permeable material and the annular outer zone 194 can be made of relatively non-permeable material, such that the outer zone 194 will remain fastened when the apparatus is wetted while the central zone 192 provides more comfort. In FIG. 19, a carrier 200 includes two circular central zones 202 of relatively thicker material, such as foam, and an outer zone 204 of relatively thin material, such as a thin film. Other properties to be varied include but are not limited to conductivity, flexibility and rigidity.

Referring to FIGS. 20 and 21, a carrier 222 of an adhesive attachment apparatus 220 constructed in accordance with the present invention can be adapted to substantially match an outer perimeter of a bottom external surface 214 of a housing 212 of a device 210 attached by the apparatus 220. In the exemplary embodiment of FIGS. 20 and 21, the device 210 includes a extending portion 218 from which a transcutaneous access tool, such as a soft cannula 216, exits the device 210. As shown, the carrier 222 is adapted to cover the extending portion 218 of the device 210 and can include a passage area for allowing passage of the cannula 216 to a patient's skin.

In FIGS. 22 and 23, a carrier 242 of an adhesive attachment apparatus 240 constructed in accordance with the present invention is also adapted to substantially match an outer perimeter of a bottom external surface 234 of a housing 232 of a device 230 attached by the apparatus 240. In the exemplary embodiment of FIGS. 22 and 23, the device 230 includes a recessed portion 238 from which a transcutaneous access tool, such as a soft cannula 236, exits the device 230. As shown, the carrier 242 is adapted to have a corresponding recessed portion to match the recessed portion 238 of the device 230.

Referring to FIGS. 24, 26 and 27, a carrier 252 of an adhesive attachment apparatus 250 constructed in accordance with the present invention includes a portion 254 comprised of a material that physically or chemically changes upon contacting a predetermined fluid. In one exemplary embodiment, wherein the apparatus 250 is intended for use with a fluid delivery device 260 for delivering insulin, the portion 254 of the carrier 252 can be comprised of material that physically or chemically changes upon contacting insulin. In this manner, the portion 254 of the carrier 252 can be used to determine if a cannula 262 of the fluid delivery device 260 is undesirably leaking fluid between the carrier 252 and the skin surface of a patient. Such leakage may occur, for example, if the cannula 262 has been prematurely pulled out of the patient's skin. The physical or chemical change can include a change in color of the portion 254 of the carrier 252, as shown in FIG. 27. Alternatively, the physical or chemical change can include a change in thickness of the portion 254 of the carrier 252, a change in electrical conductivity, or a change in mass or density of the carrier. The skin adhesive layer of the adhesive attachment apparatus 250 can also be provided with a material that physically or chemically changes upon contacting a predetermined fluid.

The fluid delivery device 260 of FIGS. 24 and 25, which is similar to the fluid delivery device of FIGS. 1 through 3, is provided with sensors 264, 266 for detecting a physical or chemical change in the indicating portion 254 of the carrier 252 upon a fluid leak. In the exemplary embodiment of FIGS. 24 and 25, the sensors comprise a light transmitter 264 and a light receiver 266 positioned above a transparent portion 268 of a housing 270 of the fluid delivery device 260. The transparent portion 268 is located at the exit port of the cannula 262, and the indicating portion 254 of the carrier 252 is positioned below the transparent portion 268. Accordingly, when the indicating portion 254 of the carrier 252 changes color upon a fluid leak from the cannula 262, the light transmitter 264 and the light receiver 266 positioned above the transparent portion 268 are used to detect the color change. The light transmitter 264 and the light receiver 266 can in turn be connected to the local processor of the fluid delivery device 260, which can be programmed to activated an alarm or transmit a signal upon detecting a fluid leak. Alternatively, a mechanical switch, strain gauge or other sensor can be integrated into fluid delivery device 260 such that when indicating portion 254 changes physical or chemical property in the presence of a fluid leak, such as an increase in mass, appropriate information is sent to the local processor.

In FIG. 28, a carrier 272 of an adhesive attachment apparatus 270 constructed in accordance with the present invention is provided with a tab 273 extending therefrom. The tab 273 is free of adhesive and is provided as an aid in removing the adhesive attachment apparatus 270 from a patient's skin when the patient is finished using the apparatus. Skin adhesive layers 278, 280 of the apparatus 270 include discontinuous segments 280 extending away from the tab 273, which are adapted to aid in removing the carrier 272 using the tab 273. The discontinuous segments 280 of adhesive secure the carrier 272 to the patient's skin during use, but do not interfere with removing the carrier 272 using the tab 273.

In FIG. 29, a carrier 292 of an adhesive attachment apparatus 290 constructed in accordance with the present invention is provided with a plurality of user-removable, stacked layers 293a, 293b. In the exemplary embodiment of FIG. 29, the carrier 292 is provided with two user-removable, stacked layers 293a, 293b. The carrier 292, however, can be provided with just one, or more than two of the user-removable, stacked layers 293a, 293b. Although not shown, each user-removable layer 293a, 293b is separated by an intermediate adhesive layer that is provided on a lower surface of the layer. Preferably, surface areas of the intermediate adhesive layers are less than respective areas of surfaces of the stacked layers 293a, 293b.

The user-removable layers 293a, 293b of the carrier 292 allow a patient to control the depth that a cannula 17 of the fluid delivery device 10 will extend into the skin of the patient. For example, if a patient is above average in weight, it may be desirable for the cannula 17 to extend further into the patient's skin. In contrast, if a patient is below average in weight, it may be preferably for the cannula 17 not to extend too far into the patient's skin so that the cannula 17 will not be so uncomfortable when deployed in the patient's skin for an extended period of time. By successively removing the user-removable layers 293a, 293b, the depth that the cannula 17 will extend into the patient's skin increases. In one exemplary embodiment, the layers 293a, 293b are provided in different colors so that a patient can visually determine the insertion depth create by the removal of each layer. In another exemplary embodiment, the layers 293a, 293b of the carrier 290 are labeled. In other exemplary embodiments, the layers 293a, 293b of the carrier 290 are provided with different properties, e.g. different adhesive strength, different thickness, etc.

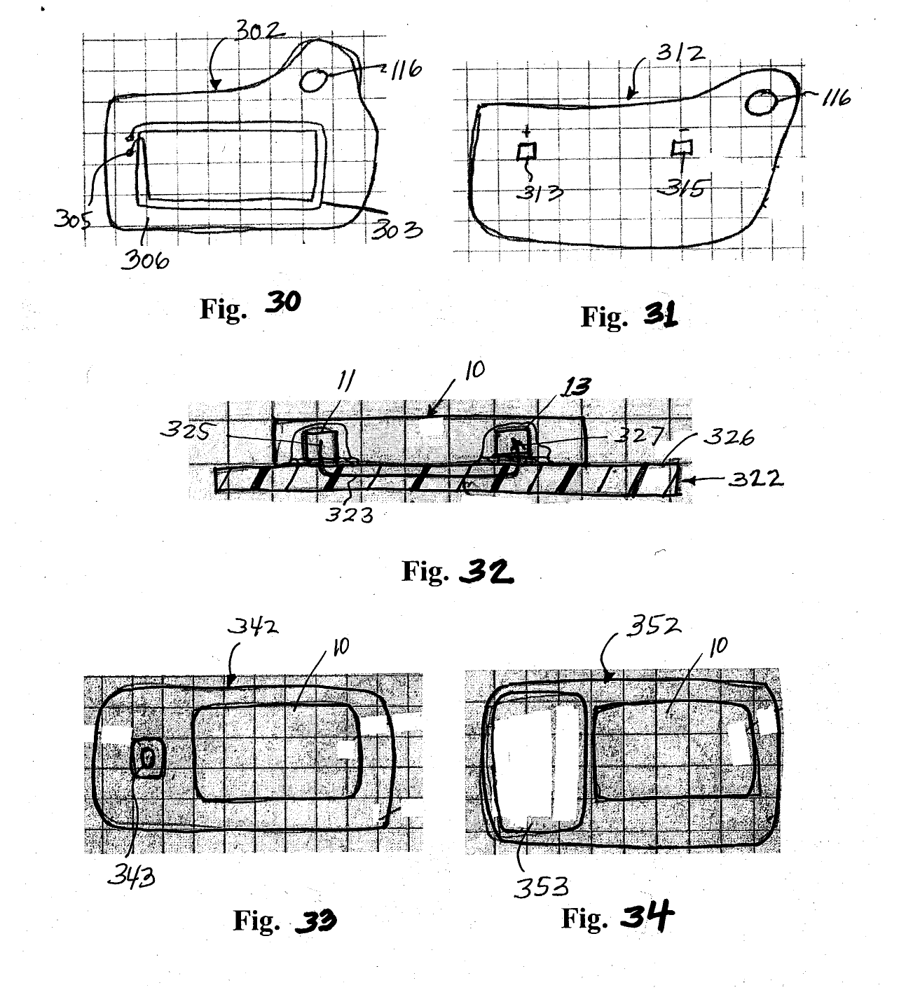

Referring now to FIGS. 30 through 34, carriers of adhesive attachment apparatuses constructed in accordance with the present invention can be provided with accessories. The accessories can be specifically provided for use as part of a fluid delivery secured to a patient by the apparatuses, or can simply be provided for convenience. In the exemplary embodiment of FIG. 30, for example, an antenna 303 is embedded in a carrier 302 and includes at least one end extending 305 from a device-contacting surface 306 of the carrier 302 for connection to a fluid delivery device to be secured to the carrier. In the exemplary embodiment of FIG. 31, sensors 313, 315 are embedded in a carrier 312 and are adapted to communicate with a fluid delivery device secured to the carrier 312. In one exemplary embodiment, the sensors 313, 315 comprise at least one of a temperature sensor and an impedance sensor for providing an indication of whether the skin-contacting surface of the carrier 312 remains in contact with a patient's skin upon the apparatus being attached to a patient's skin, or one or more other functional or physiologic parameters.

In the exemplary embodiment of FIG. 32, a conductor 323 is embedded in a carrier 322 and includes opposing ends 325, 327 extending from a device-contacting surface 326 of the carrier for mating with receptacles 11, 13, respectively, of a fluid delivery device 10 secured to the carrier 322. In this manner, an electrical circuit within the device 10 is closed upon attachment of the device 10 to the carrier 322. Alternatively, a device contacting adhesive of the carrier can be made electrically conductive in order to close a circuit within a device attached to the carrier. Although not shown, a Faraday cage of conductive screen mesh can also be provided on the carrier to help shield electronics of the attached fluid delivery device from electromagnetic interference.

In the exemplary embodiment of FIG. 33, an attachment mechanism, such as a clip 343, is secured to a portion of a carrier 342 extending beyond a perimeter of an attached fluid delivery device 10. The attachment mechanism can be used to attach other devices or accessories to the carrier 342. In the exemplary embodiment of FIG. 34, a label 353 is secured to the carrier 352. The label 353 can include information about the fluid delivery device 10, such as information about the type of medication contained in the device 10 or allergy information. The label 353 may also contain text that is readable by an X-ray device, such that a person wearing the carrier 342 and the attached device 10 can pass through an X-ray scanner and provide sufficient information to prevent alarming an attendant. In the embodiment where the label 353 contains X-ray readable information, it is not necessary for label 353 to extend beyond the perimeter of the attached device.

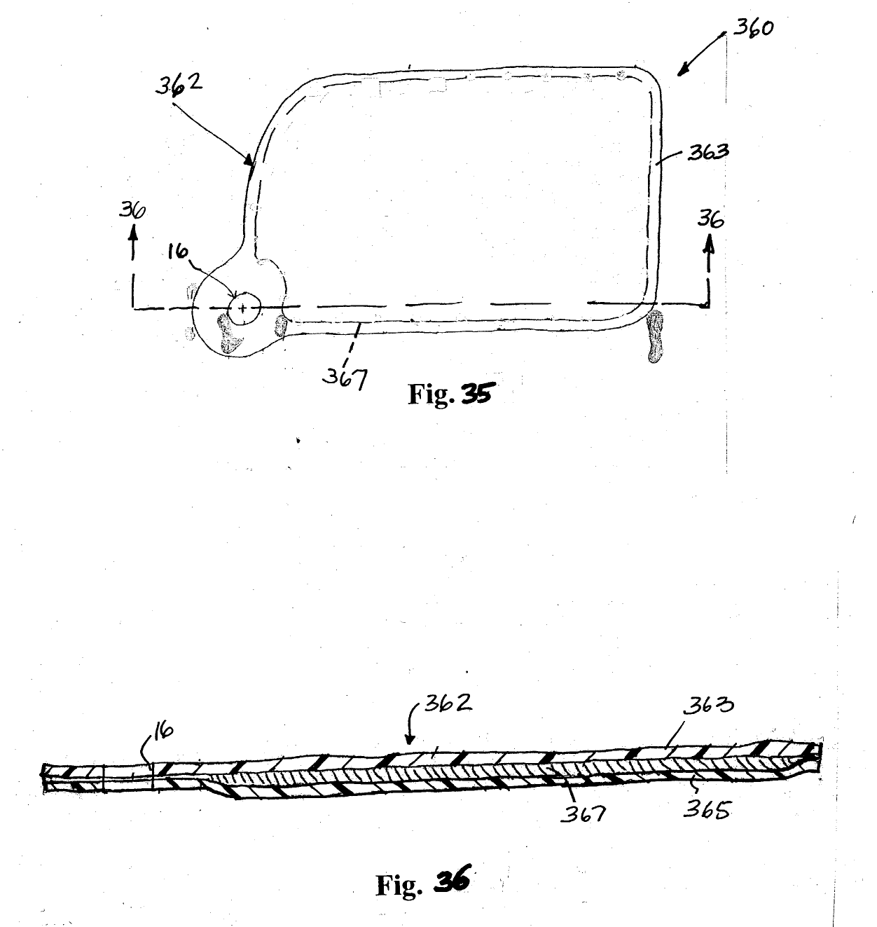

Referring to FIGS. 35 and 36, a carrier 362 of an adhesive attachment apparatus 360 constructed in accordance with the present invention includes first and second layers 363, 365 separated by a bladder 367. The bladder 367 provides an added layer of cushioning and flexibility to the carrier 362. The bladder may also serve as an insulator, isolating the internal components, such as a liquid medication, from body heat. In the exemplary embodiment of FIGS. 35 and 36, the bladder 367 contains a gel. Alternatively, however, the bladder 367 can contain another suitable material, such as air or water for example.

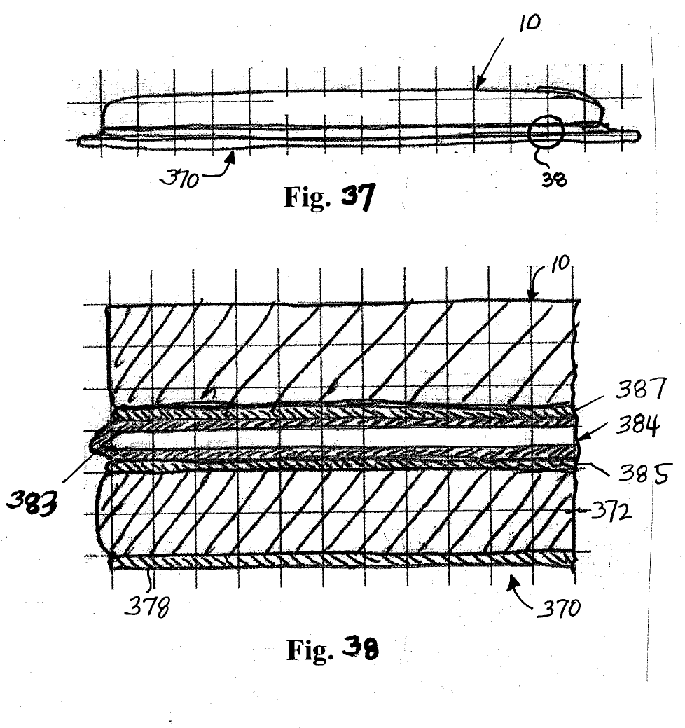

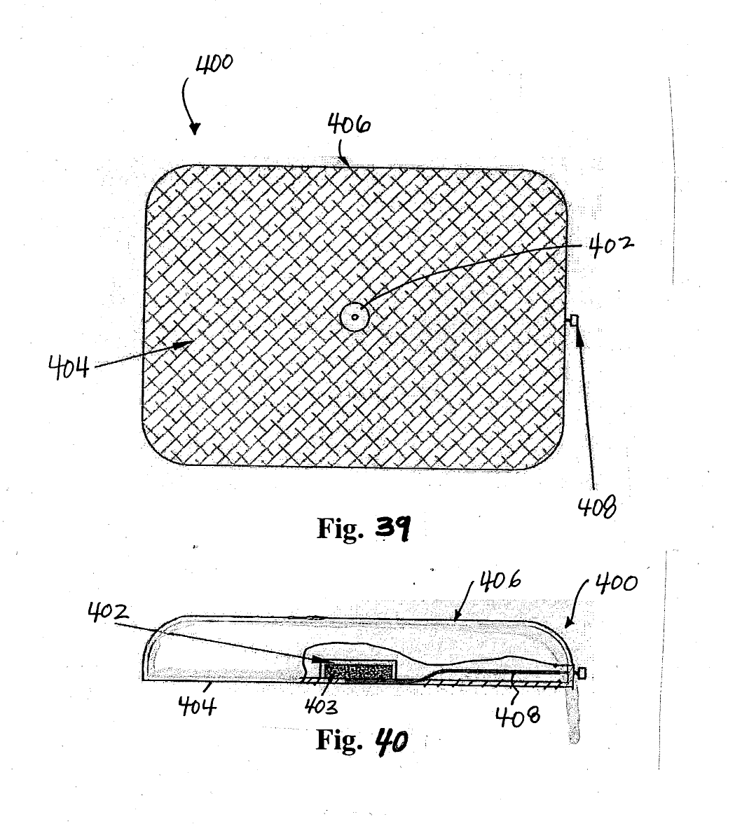

Another exemplary embodiment of a carrier 372 of an adhesive attachment apparatus 370 constructed in accordance with the present invention is shown in FIGS. 37 and 38. The adhesive attachment apparatus 370 includes a skin adhesive layer 378 for securing the carrier 372 to a patient's skin, and a device adhesive layer 384 securing the carrier 372 to a device, such as a fluid delivery device 10. The device adhesive layer 384 includes first and second layers 385, 387 separated by a bladder 383. The bladder 382 provides an added layer of cushioning and flexibility to the adhesive attachment apparatus 370. The bladder may also serve as an insulator, isolating the internal components, such as a liquid medication, from body heat. In the exemplary embodiment of FIGS. 37 and 38, the bladder 383 contains a gel. Alternatively, however, the bladder 383 can contain another suitable material, such as air or water for example.

Referring now to FIGS. 39 and 40, the present invention also provides a device 400 adapted for attachment to an adhesive attachment apparatus, such as the adhesive attachment apparatus 100 of FIGS. 1 through 3. In one exemplary embodiment, the device 400 can comprise a fluid delivery device, such as the fluid delivery device 10 of FIGS. 1 through 3. The device of FIGS. 39 and 40, however, further includes a compartment 402 positioned adjacent a skin-contacting surface 404 of a housing 406 of the device 400, and a release mechanism 408 extending from the compartment 402 for opening the compartment 402. The compartment 402 is preferably filled with a glue remover 403 (e.g., UniSolve™ glue remover), so that upon being released from the compartment 402 the glue remover 403 dissolves the adhesive layers of the adhesive attachment apparatus and makes removal of the adhesive attachment apparatus from a patient's skin easier. The compartment is preferably located in a central location of housing 406. In the exemplary embodiment shown, the release mechanism comprises a cord 408 extending from the compartment 402 and which is adapted to open the compartment 402 upon being pulled. Alternatively, the release mechanism can be electrically activated by a control from the local processor, such as via a command received from a remote control device. As another alternative, an adhesive attachment apparatus constructed in accordance with the present invention can include its own glue remover compartment and release mechanism.

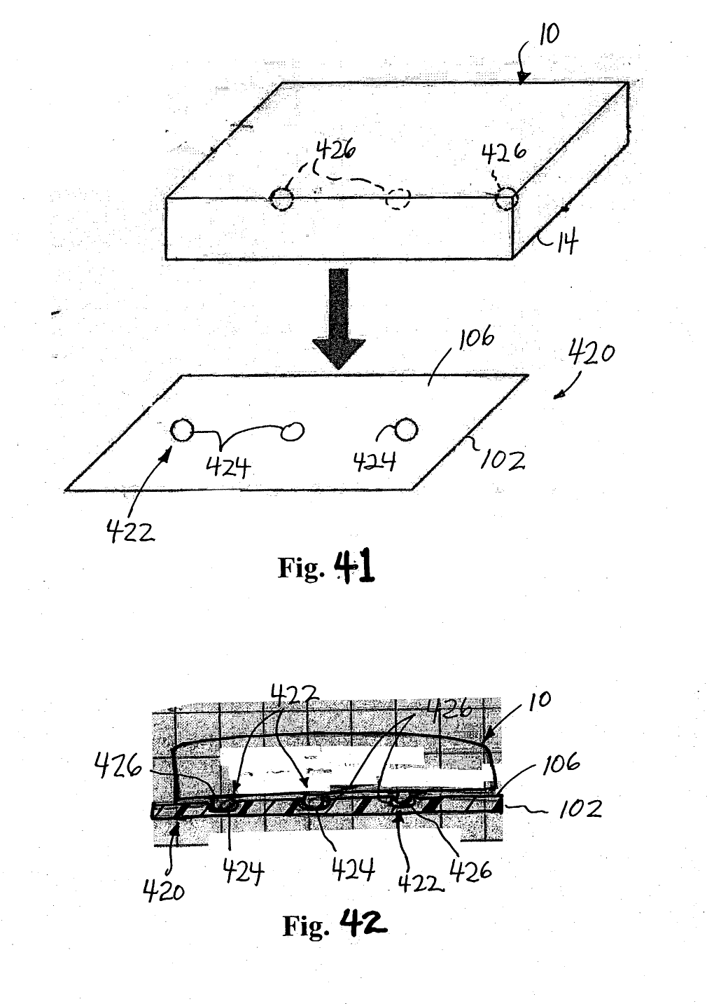

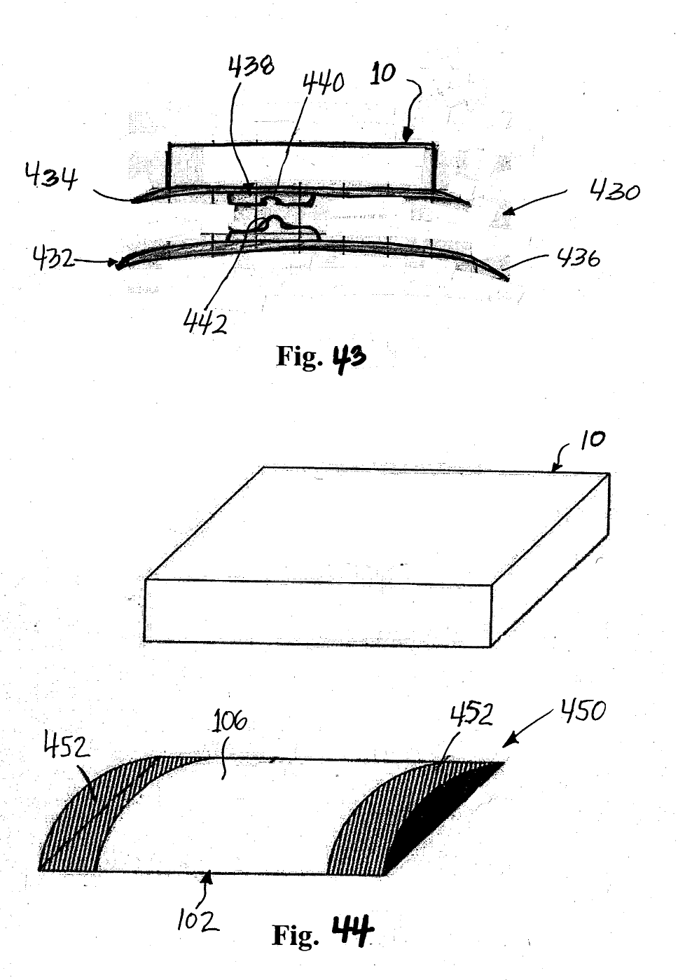

Referring now to FIGS. 41 and 42, another exemplary embodiment of an adhesive attachment apparatus 420 constructed in accordance with the present invention is shown. The apparatus 420 is similar to the apparatus 100 of FIGS. 1 through 5, such that similar elements have the same reference numerals. In the apparatus 420 of FIGS. 41 and 42, however, the means for securing the device-contacting surface 106 of the carrier 102 against the bottom external surface 14 of the device 10 includes at least one mechanical connector 422. In the exemplary embodiment of FIGS. 41 and 42, three of the mechanical connectors 422 are provided and each includes a socket 424 fixed to the device-contacting surface 106 of the carrier 102 and receiving a stud 426 fixed to the bottom external surface 14 of the device 10, in a snap-fit manner. The mechanical connectors 422 provide a method of removably attaching the device 10 to the carrier 102. In an alternative embodiment, the studs can be fixed to the carrier 102 and the sockets can be fixed to the device 10.

Another exemplary embodiment of a carrier 432 of an adhesive attachment apparatus 430 constructed in accordance with the present invention is shown in FIG. 43. The carrier 432 includes a first layer 434 secured to a device 10 and a second layer 436 for being secured to a patient's skin. The first and the second layers 434, 436 are connected by a mechanical connector 438. In the exemplary embodiment shown, the mechanical connector 438 includes a socket 440 engaging a stud 442 in a snap-fit manner. The socket 440 is secured to the first layer 434 and the stud 442 is secured to the second layer 436 of the carrier 432.

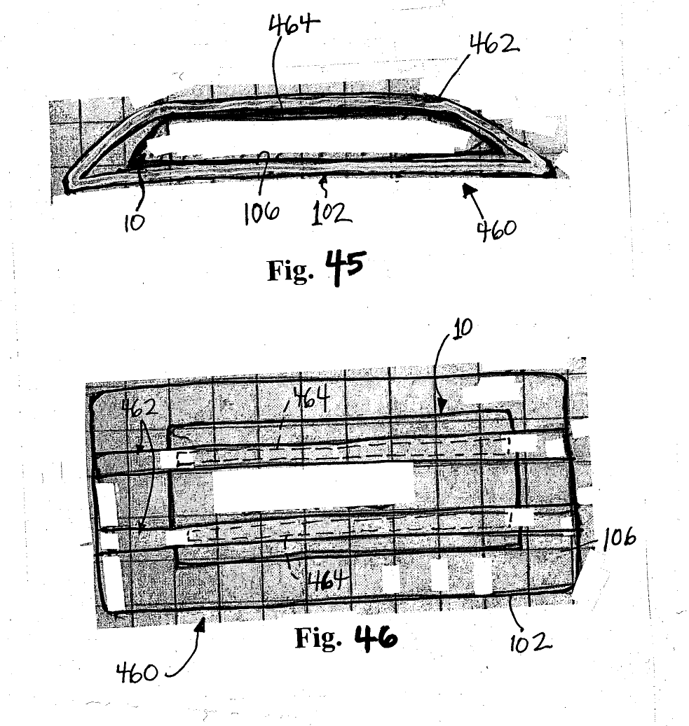

Referring to FIG. 44, a further exemplary embodiment of an adhesive attachment apparatus 450 constructed in accordance with the present invention is shown. The apparatus 450 is similar to the apparatus 100 of FIGS. 1 through 5, such that similar elements have the same reference numerals. In the apparatus 450 of FIG. 44, however, the means for securing the device-contacting surface 106 of the carrier 102 against the bottom external surface 14 of the device 10 includes at least one pocket 452 secured to the carrier 102 for receiving the device 10 therein and holding the device against the device-contacting surface 106 of the carrier. In the exemplary embodiment of FIG. 44, the apparatus 450 includes two pockets 452 positioned at opposite ends of the carrier 102.

Referring to FIGS. 45 and 46, an additional exemplary embodiment of an adhesive attachment apparatus 460 constructed in accordance with the present invention is shown. The apparatus 460 is similar to the apparatus 100 of FIGS. 1 through 5, such that similar elements have the same reference numerals. In the apparatus 460 of FIGS. 45 and 46, however, the means for securing the device-contacting surface 106 of the carrier 102 against the bottom external surface 14 of the device 10 includes straps 462 connected to the carrier 102 for passing around the device 10 and holding the device against the device-contacting surface 106 of the carrier 102. As shown, the straps 462 extends between ends of the carrier 102. In addition, each strap 462 includes a layer of adhesive 464 securing the strap to the device 10.

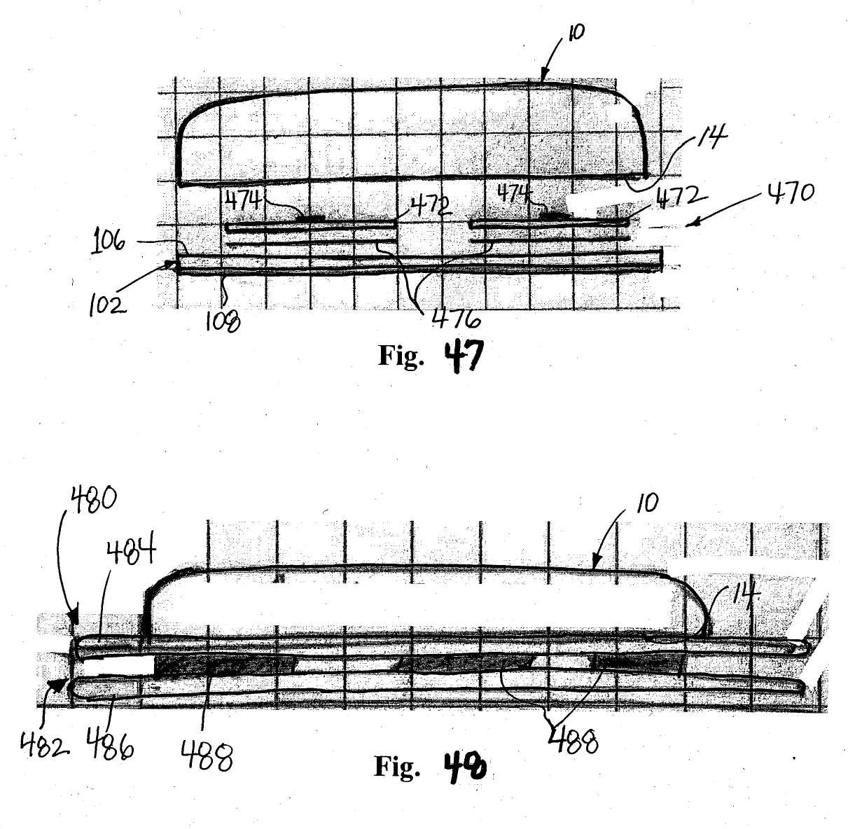

Referring to FIG. 47, a further exemplary embodiment of an adhesive attachment apparatus 470 constructed in accordance with the present invention is shown. The apparatus 470 is similar to the apparatus 100 of FIGS. 1 through 5, such that similar elements have the same reference numerals. In the apparatus 470 of FIG. 47, however, the means for securing the device-contacting surface 106 of the carrier 102 against the bottom external surface 14 of the device 10 includes intermediate layers 472 of flexible material secured to the device-contacting surface 106 of the carrier 102, and device adhesive layers 474 secured to the intermediate layers 472 of flexible material for adhesion against the bottom external surface 14 of the device 10. In addition, the intermediate layers 472 of flexible material are secured to the device-contacting surface 106 of the carrier 102 by intermediate layers 476 of adhesive.

Another exemplary embodiment of a carrier 482 of an adhesive attachment apparatus 480 constructed in accordance with the present invention is shown in FIG. 48. The carrier 482 includes a first layer 484 secured to a device 10 and a second layer 486 for being secured to a patient's skin. The first and the second layers 484, 486 are connected by resiliently flexible spacers 488.

Referring to FIGS. 49 through 51, the present invention also provides devices 500, 500′, 500″ that are adapted for attachment to a patient's skin surface. In one exemplary embodiment, the devices 500, 500′, 500″ can each comprise a fluid delivery device, such as the fluid delivery device 10 of FIGS. 1 through 3. The devices 500, 500′, 500″ of FIGS. 49 through 51, however, each further include a housing 502 including an external surface 504 and feet 506 extending from the external surface 504 to distal surfaces 508 thereof and a skin adhesive layer 510 secured to the distal surface of each foot for securing the devices to a skin surface of a patient.

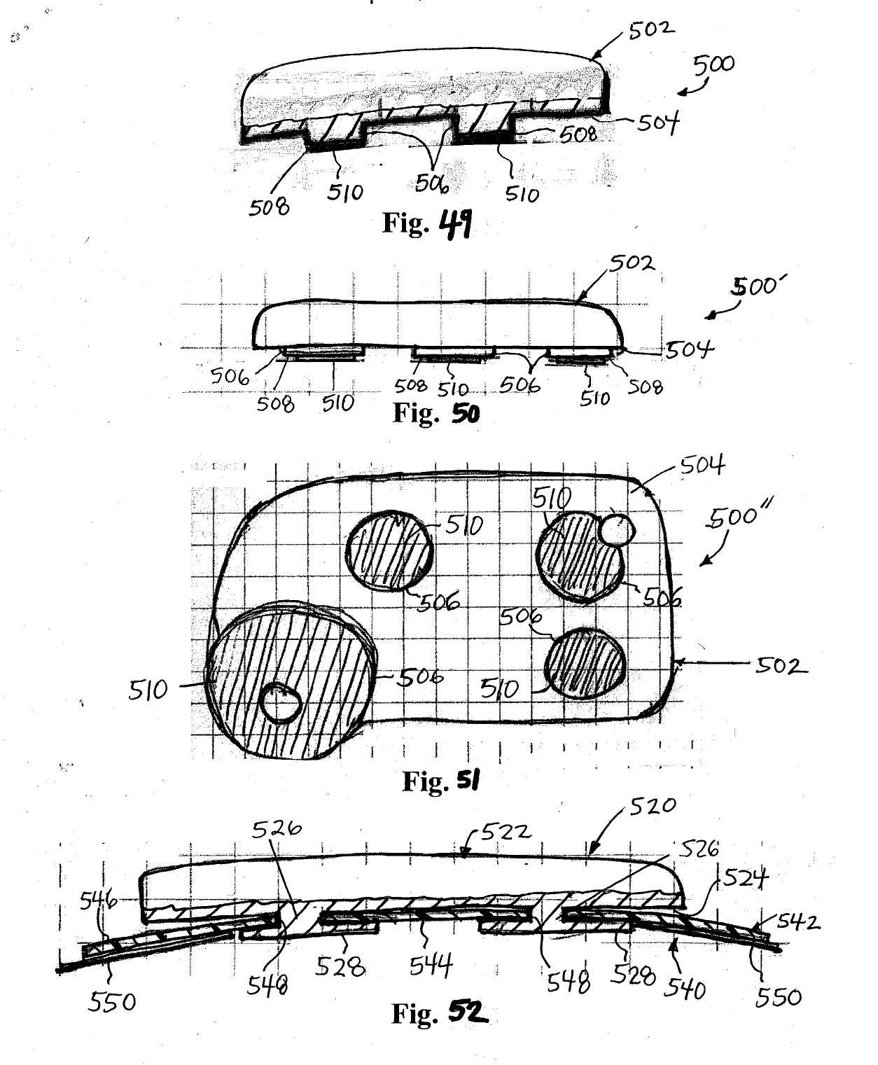

In the embodiment 500 of FIG. 49, surface areas of the skin adhesive layers 510 are substantially equal to areas of the distal surfaces 508 of the feet 506 of the housing 502. In the embodiment 500′ of FIG. 50, surface areas of the skin adhesive layers 510 are less than the areas of the distal surfaces 508 of the feet 506 of the housing 502. In the embodiment 500″ of FIG. 51, surface areas of the skin adhesive layers 510 are substantially equal to areas of the distal surfaces 508 of the feet 506 of the housing 502. In addition, in the embodiment 500″ of FIG. 51, one of the feet 506 of the housing 502 defines an external outlet port 512 of the housing and the adhesive layer 510 continuously surrounds the port 512, and the housing includes an external inlet port 514 positioned adjacent at least one of the feet 506 of the housing 502.

In FIG. 52, additional exemplary embodiments of a device 520 and an adhesive attachment apparatus 540 constructed in accordance with the present invention are shown. In one exemplary embodiment, the device 520 can comprise a fluid delivery device, such as the fluid delivery device 10 of FIGS. 1 through 3. The device 520 of FIG. 52, however, further includes a housing 522 having an external surface 524 and legs 526 extending from the external surface to distal overhanging feet 528. A carrier 542 of the adhesive attachment apparatus 540 has a skin-contacting surface 544, and an opposed device-contacting surface 546. The carrier 542 is positioned between the external surface 524 of the housing 522 and the overhanging feet 528 and has openings 548 receiving the legs 526, such that the overhanging feet secure the carrier 542 to the device 520. The adhesive attachment apparatus 540 also include skin adhesive layers 550 secured to the skin-contacting surface 544 of the carrier 542 for securing the carrier and the attached device 520 to a skin surface of a patient. In the exemplary embodiment shown, a total surface area of the skin adhesive layers 550 is less than an area of the skin-contacting surface 544 of the carrier 542.

As illustrated by the above described exemplary embodiments, the present invention generally provides new and improved skin attachment apparatuses and methods for attaching a device for delivering fluid, such as insulin for example, to a patient. It should be understood that the embodiments described herein are merely exemplary and that a person skilled in the art may make variations and modifications to the embodiments described without departing from the spirit and scope of the present invention. All such equivalent variations and modifications are intended to be included within the scope of this invention as defined by the appended claims.

What is claimed is:

1. An apparatus adapted to be disposed between a rigid or semi-rigid device and human skin for reliably attaching the device to the skin for an extended period of time, the system comprising:
a carrier having a skin-contacting surface, and an opposed device-contacting surface;
means for securing the device-contacting surface of the carrier against a bottom external surface of a device attached by the apparatus; and
at least one skin adhesive layer secured to the skin-contacting surface of the carrier for securing the carrier to the skin, wherein a surface area of the skin adhesive layer is less than an area of the skin-contacting surface.

2. An apparatus according to claim 1, wherein the carrier includes a passage area for passage of a portion of a device attached by the apparatus between the device-contacting surface and the skin-contacting surface, and the skin adhesive layer comprises a layer of adhesive positioned around the passage area of the carrier.

3. An apparatus according to claim 1, wherein the carrier includes a passage area comprising an opening extending between the device-contacting surface and the skin-contacting surface for passage of a portion of a device attached by the apparatus, and the skin adhesive layer comprises a continuous layer of adhesive positioned around the opening in the carrier.

4. An apparatus according to claim 1, wherein the skin adhesive layer comprises a continuous layer of adhesive positioned adjacent an outer edge of the carrier.

5. An apparatus according to claim 1, wherein the carrier is adapted to extend beyond a perimeter of a bottom external surface of a device attached by the apparatus.

6. An apparatus according to claim 1, wherein the carrier is adapted to substantially match a perimeter of a bottom external surface of a device attached by the apparatus.

7. An apparatus according to claim 1, wherein the skin adhesive layer comprises pressure sensitive adhesives.

8. An apparatus according to claim 1, wherein the skin adhesive layer comprises separate zones of different properties.

9. An apparatus according to claim 8, wherein the separate zones of the skin adhesive layer comprise at least a zone of relative weak adhesive and a zone of relatively strong adhesive.

10. An apparatus according to claim 9, wherein the carrier includes a passage area for passage of a portion of a device attached by the apparatus between the device-contacting surface and the skin-contacting surface, and the zone of relatively strong adhesive is located closer to the passage area than the zone of relative weak adhesive.

11. An apparatus-according to claim 8, wherein the separate zones of the skin adhesive layer comprise at least one zone of water-resistant adhesive.

12. An apparatus according to claim 1, wherein the skin adhesive layer is annular.

13. An apparatus according to claim 1, wherein the skin adhesive layer comprises an annular array of discontinuous segments.

14. An apparatus according to claim 1, wherein the skin adhesive layer includes discontinuous segments.

15. An apparatus according to claim 1, wherein the carrier is comprised of resiliently flexible material.

16. An apparatus according to claim 1, wherein the carrier is comprised of a foam plastic.

17. An apparatus according to claim 1, wherein the carrier is comprised of a closed cell foam plastic.

18. An apparatus according to claim 1, wherein the carrier is impregnated with an agent.

19. An apparatus according to claim 18, wherein the agent comprises a disinfectant.

20. An apparatus according to claim 18, wherein the agent comprises a skin moisturizer.

21. An apparatus according to claim 18, wherein the agent comprises an anti-inflammatory.

22. An apparatus according to claim 18, wherein the agent comprises an antibacterial solution.

23. An apparatus according to claim 1, wherein the carrier comprises a transdermal drug delivery device.

24. An apparatus according to claim 23, wherein the drug delivery device contains a therapeutic diabetes agent.

25. An apparatus according to claim 1, wherein the carrier comprises user-removable portions.

26. An apparatus according to claim 25, wherein the user-removable portions of the carrier comprise successive outer peripheral rings of the carrier.

27. An apparatus according to claim 1, wherein the carrier comprises separate zones of different properties.

28. An apparatus according to claim 27, wherein the separate zones of the carrier include a zone of relatively thick material and a zone of relatively thin material.

29. An apparatus according to claim 27, wherein the separate zones of the carrier include a zone of relatively porous material and a zone of relatively non-porous material.

30. An apparatus according to claim 27, wherein the separate zones of the carrier include a first zone peripherally surrounded by a second zone.

31. An apparatus according to claim 30, wherein the second zone of the carrier has a thickness that decreases further from the first zone of the carrier.

32. An apparatus according to claim 27, wherein the separate zones of the carrier include first and second spaced-apart zones peripherally surrounded by a third zone.

33. An apparatus according to claim 32, wherein the third zone of the carrier has a thickness that decreases further from the first and the second zones of the carrier.

34. An apparatus according to claim 1, wherein the carrier comprises a material that physically changes upon contacting a predetermined fluid.

35. An apparatus according to claim 34, wherein the physical change comprises a change in color.

36. An apparatus according to claim 34, wherein the physical change comprises at least one of a change in thickness and a change in electrical conductivity.

37. An apparatus according to claim 1, wherein the skin adhesive layer comprises a material that physically changes upon contacting a predetermined fluid.

38. An apparatus according to claim 37, wherein the predetermined fluid comprises insulin.

39. An apparatus according to claim 37, wherein the physical change comprises a change in color.

40. An apparatus according to claim 1, wherein the skin adhesive layer comprises user-removable portions.

41. An apparatus according to claim 41, wherein the user-removable portions of the skin adhesive layer comprise successive outer peripheral rings of the skin adhesive layer.

42. An apparatus according to claim 1, wherein the carrier includes a tab extending therefrom, and the skin adhesive layer includes discontinuities extending towards the tab.

43. An apparatus according to claim 1, wherein the carrier comprises a plurality of removable stacked layers, and wherein each layer is separated by an intermediate adhesive layer, wherein surface areas of the intermediate adhesive layers are less than areas of surfaces of the stacked layers.

44. An apparatus according to claim 43, wherein the layers of the carrier are labeled and/or provided in different colors.

45. An apparatus according to claim 1, further comprising a removable protective layer covering the skin adhesive layer.

46. An apparatus according to claim 1, wherein an antenna is embedded in the carrier and includes at least one end extending from the device-contacting surface.

47. An apparatus according to claim 1, wherein a conductor is embedded in the carrier and includes opposing ends extending from the device-contacting surface.

48. An apparatus according to claim 1, wherein at least one sensor is embedded in the carrier.

49. An apparatus according to claim 48, wherein the sensor is adapted to provide an indication of whether the skin-contacting surface of the carrier remains in contact with a patient's skin upon the apparatus being attached to a patient's skin.

50. An apparatus according to claim 49, wherein the sensor comprises a temperature sensor.

51. An apparatus according to claim 49, wherein the sensor comprises a impedance sensor.

52. An apparatus according to claim 1, further comprising an attachment mechanism secured to the carrier.

53. An apparatus according to claim 1, wherein the attachment mechanism comprises a clip.

54. An apparatus according to claim 1, further comprising a label secured to the carrier.

55. An apparatus according to claim 1, wherein the carrier comprises first and second layers separated by a bladder.

56. An apparatus according to claim 55, wherein the bladder contains at least one of a gel, foam, fluid, air or powder.

57. An apparatus according to claim 34, wherein the predetermined fluid comprises insulin.

58. An apparatus according to claim 1, wherein the carrier comprises at least two layers connected by resiliently flexible spacers.

59. An apparatus according to claim 8, wherein the separate zones of the skin adhesive layer comprise at least a zone of relative thin adhesive and a zone of relatively thick adhesive.

60. An apparatus according to claim 59, wherein the carrier includes a passage area for passage of a portion of a device attached by the apparatus between the device-contacting surface and the skin-contacting surface, and the zone of relatively thick adhesive is located closer to the passage area than the zone of relative thin adhesive.

61. An apparatus according to claim 1, wherein the means for securing the device-contacting surface of the carrier against a bottom external surface of a device attached by the apparatus comprises at least one device adhesive layer secured to the device-contacting surface of the carrier.

62. An apparatus according to claim 61, wherein a surface area of the device adhesive layer is less than an area of the device-contacting surface.

63. An apparatus according to claim 61, wherein the carrier includes a passage area for passage of a portion of a device attached by the apparatus between the device-contacting surface and the skin-contacting surface, and the device adhesive layer comprises an first annular layer of adhesive secured to the device-contacting surface of the carrier and positioned around the passage area of the carrier.

64. An apparatus according to claim 63, wherein the device adhesive layer further comprises a second layer of adhesive spaced from the first layer.

65. An apparatus according to claim 61, wherein the device adhesive layer comprises first and second layers separated by a bladder.

66. An apparatus according to claim 66, wherein the bladder contains air.

67. An apparatus according to claim 61, wherein the means for securing the device-contacting surface of the carrier against a bottom external surface of a device further comprises a strap connected to the carrier for passing around the device and holding the device against the device-contacting surface of the carrier.

68. An apparatus according to claim 1, wherein the means for securing the device-contacting surface of the carrier against a bottom external surface of a device attached by the apparatus comprises at least one strap connected to the carrier for passing around the device and holding the device against the device-contacting surface of the carrier.

69. An apparatus according to claim 68, wherein the strap extends between ends of the carrier.

70. An apparatus according to claim 68, further comprising a layer of adhesive secured to the strap for securing the strap to a device attached by the apparatus.

71. An apparatus according to claim 1, wherein the means for securing the device-contacting surface of the carrier against a bottom external surface of a device attached by the apparatus comprises at least one pocket secured to the carrier for receiving the device therein and holding the device against the device-contacting surface of the carrier.

72. An apparatus according to claim 71, wherein the pocket is positioned at an end of the carrier.

73. An apparatus according to claim 71, including two of the pockets positioned at opposite ends of the carrier.

74. An apparatus according to claim 1, wherein the means for securing the device-contacting surface of the carrier against a bottom external surface of a device attached by the apparatus comprises a mechanical connector.

75. An apparatus according to claim 74, wherein the mechanical connector comprises at least one socket for receiving a stud of the device in a snap-fit manner.

76. A system including the apparatus of claim 75, and further including a device having a stud received by the socket of the apparatus in a snap-fit manner.

77. An apparatus according to claim 74, wherein the mechanical connector comprises at least one stud for being received in a socket of the device in a snap-fit manner.

78. A system including the apparatus of claim 77, and further including a device having a socket receiving the stud of the apparatus in a snap-fit manner.

79. An apparatus according to claim 1, wherein the carrier comprises at least two layers connected by a mechanical connector.

80. An apparatus according to claim 79, wherein the mechanical connector comprises a socket secured to one of the layers of the carrier receiving a stud secured to the other of the layers of the carrier in a snap-fit manner.

81. An apparatus according to claim 1, wherein the means for securing the device-contacting surface of the carrier against a bottom external surface of a device attached by the apparatus comprises:
at least one intermediate layer of flexible material secured to the device-contacting surface of the carrier; and
at least one device adhesive layer secured to the intermediate layer of flexible material for adhesion against a bottom external surface of a device attached by the apparatus.

82. An apparatus according to claim 81, wherein the intermediate layer of flexible material is secured to the device-contacting surface of the carrier by an intermediate layer of adhesive.

83. An apparatus according to claim 81, wherein the intermediate layer of flexible material comprises two spaced-apart intermediate layers of flexible material.

84. A system including the apparatus of claim 1, and further including a device secured to the device-contacting surface of the carrier.

85. A system according to claim 84, wherein the device includes:
a compartment positioned adjacent a bottom surface of the device and containing adhesive remover; and
a release mechanism extending from the compartment for opening the compartment.

86. A system according to claim 85, wherein the release mechanism is manually operative.

87. An apparatus according to claim 1, further comprising:
a compartment positioned adjacent the skin contacting surface of the carrier and containing adhesive remover; and
a release mechanism extending from the compartment for opening the compartment.

88. An apparatus according to claim 87, wherein the release mechanism is manually operative.

89. A device for delivering fluid to a patient, comprising:
an exit port assembly adapted to connect to a transcutaneous patient access tool;
a dispenser for causing fluid from a reservoir to flow to the exit port assembly;
a local processor connected to the dispenser and programmed to cause a flow of fluid to the exit port assembly based on flow instructions;
a wireless receiver connected to the local processor for receiving flow instructions from a separate, remote control device and delivering the flow instructions to the local processor;
a housing containing the exit port assembly, the dispenser, the local processor, and the wireless receiver;
a compartment positioned adjacent a skin-contacting surface of the housing and containing adhesive remover; and
a release mechanism extending from the compartment for opening the compartment.

90. A device according to claim 89, wherein the release mechanism is manually operative.

91. A device according to claim 89, wherein the housing is free of user input components for providing flow instructions to the local processor.

92. A device for delivering fluid to a patient, comprising:
an exit port assembly adapted to connect to a transcutaneous patient access tool;
a dispenser for causing fluid from a reservoir to flow to the exit port assembly;
a local processor connected to the dispenser and programmed to cause a flow of fluid to the exit port assembly based on flow instructions;
a wireless receiver connected to the local processor for receiving flow instructions from a separate, remote control device and delivering the flow instructions to the local processor;
a housing containing the exit port assembly, the dispenser, the local processor, and the wireless receiver, and including,
an external surface;
feet extending from the external surface to distal surfaces; and
a separate skin adhesive layer secured to the distal surface of each foot for securing the device to a skin surface of a patient.

93. A device according to claim 92, wherein the housing is free of user input components for providing flow instructions to the local processor.

94. A device according to claim 92, wherein surface areas of the skin adhesive layers are less than an area of the distal surfaces of the feet of the housing.

95. A device according to claim 92, wherein surface areas of the skin adhesive layers are about equal to an area of the distal surfaces of the feet of the housing.

96. A device according to claim 92, wherein one of the feet of the housing defines an external outlet port of the housing and the adhesive layer continuously surrounds the port.

97. A device according to claim 92, wherein the housing includes an external inlet port positioned adjacent to at least one of the feet of the housing.

98. A device for delivering fluid to a patient, comprising:
an exit port assembly adapted to connect to a transcutaneous patient access tool;
a dispenser for causing fluid from a reservoir to flow to the exit port assembly;
a local processor connected to the dispenser and programmed to cause a flow of fluid to the exit port assembly based on flow instructions;
a wireless receiver connected to the local processor for receiving flow instructions from a separate, remote control device and delivering the flow instructions to the local processor;
a housing containing the exit port assembly, the dispenser, the local processor, and the wireless receiver, and including,
an external surface;
legs extending from the external surface to distal overhanging feet;
a carrier having a skin-contacting surface, and an opposed device-contacting surface, wherein the carrier is positioned between the external surface of the housing and the overhanging feet and has openings receiving the legs; and
at least one skin adhesive layer secured to the skin-contacting surface of the carrier.

99. A device according to claim 98, wherein the housing is free of user input components for providing flow instructions to the local processor.

100. A device according to claim 98, wherein a surface area of the skin adhesive layer is less than an area of the skin-contacting surface of the carrier.

101. A method for reliably attaching a rigid or semi-rigid device to human skin for an extended period of time, comprising:
providing a carrier having a skin-contacting surface and an opposed device-contacting surface;
attaching the device-contacting surface of the carrier to a rigid or semi-rigid device; and
attaching a portion of a total area of the skin-contacting surface of the carrier to a skin surface of a person.

102. The method of claim 101, wherein when the device is subject to external stress, the stress is transmitted through the portion of the total area of the skin-contacting surface attached to the skin surface and distributed to a portion of the total area of the skin-contacting surface unattached to the skin surface.

103. The method of claim 101, wherein an outer perimeter of the carrier extends beyond an outer perimeter of the device.

104. The method of claim 101, wherein the portion of the total area of the skin-contacting surface attached to the skin surface is attached with a pressure sensitive adhesive.

105. The method of claim 101, wherein the attachment of the skin-contacting surface of the carrier to the skin surface occurs at a number of discrete areas.

106. The method of claim 101, wherein the device-contacting surface of the carrier is attached to a rigid or semi-rigid device with a pressure sensitive adhesive.

107. The method of claim 101, wherein a portion of a total area of the device-contacting surface of the carrier is attached to the rigid or semi-rigid device.

108. The method of claim 101, wherein the attachment of the device-contacting surface of the carrier to the adjacent surface of the device occurs at a number of discrete areas.

109. The method of claim 101, wherein substantially a total area of the device-contacting surface of the carrier is attached to the rigid or semi-rigid device.

110. The method of claim 101, wherein the carrier has viscoelastic properties approaching those of skin.

111. The method of claim 101, wherein the attachment of the carrier to the device is permanent.

112. The method of claim 101, wherein the carrier is detachable from the device.

113. The method of claim 101, wherein the attachment of the carrier to the device is accomplished by locating co-operating elements of a fastening system on the adjacent surface of the device and the device-contacting surface of the carrier, respectively.

114. The method of claim 101, wherein the device is provided with an integral needle for delivery of a substance through the skin.

115. The method of claim 101, wherein a passage area is provided in the carrier for allowing an integral needle of the device access to the skin.

116. The method of claim 101, wherein a passage area is provided in the carrier for allowing an integral needle of the device access to the skin surface and a portion of the skin contacting surface surrounding the passage area is attached to the skin surface.

117. The method of claim 101, wherein the device includes a drug reservoir.
